(19) United States
(12) Patent Application Publication
(10) Pub. No.: US 2024/0350670 A1
(43) Pub. Date: Oct. 24, 2024

(54) HYBRID PROMOTERS FOR GENE EXPRESSION IN MUSCLES AND IN THE CNS

(71) Applicants: GENETHON, EVRY-COURCOURONNES (FR); UNIVERSITE D'EVRY VAL D'ESSONNE, EVRY-COURCOURONNES (FR); INSERM (INSTITUT NATIONAL DE LA SANTÉ ET DE LA RECHERCHE MÉDICALE), PARIS (FR)

(72) Inventors: GIUSEPPE RONZITTI, FONTAINEBLEAU (FR); PAULINE VIDAL, BOUSSY SAINT ANTOINE (FR)

(21) Appl. No.: 18/294,577

(22) PCT Filed: Aug. 4, 2022
(86) PCT No.: PCT/EP2022/072028

§ 371 Date: Feb. 2, 2024

(30) Foreign Application Priority Data
Aug. 4, 2021 (EP) 21306089.0

(51) Int. Cl. A61K 48/00; C12N 15/86

(52) U.S. Cl. CPC A61K 48/0058; C12N 15/86; C12N 2750/14143; C12N 2830/008

(57) ABSTRACT

The present invention relates to novel hybrid promoters. The invention further relates to expression cassettes and vectors containing said hybrid promoters. Also disclosed herein are methods implementing these hybrid promoters, in particular methods of gene therapy.

### FIELD OF THE INVENTION

The present invention relates to hybrid promoters to drive gene expression in muscles and in the CNS. The invention further relates to expression cassettes and vectors containing said hybrid promoters. Also disclosed herein are methods implementing these hybrid promoters, in particular methods of gene therapy.

### BACKGROUND OF THE INVENTION

Neuromuscular disorders that require simultaneous targeting of muscles and central nervous system (CNS) represent one of the main challenges for in vivo based gene therapy. In particular, the high doses of vector needed to efficiently transduce those two tissues are likely to induce toxicity in the liver. Yet, insufficient transgene expression in the desired target tissues and anti-transgene immunity still represent important hurdles to achieve successful gene therapy for many diseases. Therefore, there is still a need of providing strong expression of a transgene into the cell of interest, but at low dose of vectors to prevent both potential toxicity of the vector and immune response against the vector.

Adeno-associated vector (AAV) is the vector of choice for in vivo gene therapy. The transgene expression cassette used in AAV gene therapy may comprise different elements, such as enhancers and promoters, allowing the modulation of the efficacy and specificity of expression of a gene of interest in the target cells. Constitutive promoters, such as CMV or CAG induce strong expression but lack tissue specificity and are likely to drive an immune response against the transgene.

Here, we describe the identification of hybrid promoters, which allow strong specific expression in muscles and in the CNS and a reduced targeting into the liver thereby reducing the risk of immune/toxic response. The hybrid promoters of the invention thereby allow to reduce the dose of vector that is administered, or to get a stronger expression at an equivalent dose.

### SUMMARY OF THE INVENTION

The present invention provides genetic engineering strategies implementing novel hybrid promoters having muscle/CNS specificity without targeting the liver. These hybrid promoters may be used in gene therapy of neuromuscular diseases. These novel hybrid promoters are based on the use of one or more liver-selective enhancer(s) in combination with two muscle-selective promoters. In a particular embodiment, the novel hybrid promoters are based on the combination of (i) one or a plurality of liver-selective enhancer(s), (ii) a first muscle-specific enhancer which is a CK6 promoter or a functional variant thereof and (iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof.

Surprisingly, it is herein shown that such a hybrid promoter leads to a strong expression of a transgene in muscles and in the spinal cord, while not targeting the liver.

WO 2020/208032 describes the improvement of transgene expression of a muscle-selective promoter when it is fused to one or more liver-selective enhancers. Surprisingly, it is herein shown that the combination of liver-selective enhancer(s) with a first muscle-selective promoter and a second muscle-selective promoter, in a same expression cassette, leads to a synergistic effect when compared to:
the expression resulting from the combination of liver selective enhancer(s) with the first muscle-selective promoter; and
the expression resulting from the combination of liver selective enhancer(s) with the second muscle-selective promoter.

Accordingly, a first aspect of the invention relates to a nucleic acid molecule comprising the following transcription regulatory elements, operably linked to each other:
(i) one or a plurality of liver-selective enhancer(s);
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, Acta1 promoter, MCK promoter, desmin promoter, and functional variants thereof; the second muscle-selective promoter being preferably a spC5-12 promoter, CK6 promoter, CK8 promoter, Acta1 promoter or a functional variant thereof, the second muscle-selective promoter being more preferably a spC5-12 promoter, CK6 promoter or a functional variant thereof, the second muscle-selective promoter being even more preferably a spC5-12 promoter or a functional variant thereof.

In a particular embodiment, the nucleic acid molecule comprises, in this order from 5′ to 3′:
the one or plurality of liver-selective enhancer(s), the first muscle-selective promoter, and the second muscle-selective promoter;
or
the one or plurality of liver-selective enhancer(s), the second muscle-selective promoter, and the first muscle-selective promoter.

In a particular embodiment, the first muscle-selective promoter (i.e. CK6 or a functional variant thereof) is located upstream the 5′ end of the second muscle-selective promoter. In a more particular embodiment, the CK6 promoter or a functional variant thereof is located upstream the 5′ end of the spc5.12 promoter or a functional variant thereof. In a more particular embodiment, the CK6 promoter or a functional variant thereof is located upstream the 5′ end of the CK8 promoter or a functional variant thereof. In a more particular embodiment, the CK6 promoter or a functional variant thereof is located upstream the 5′ end of a second CK6 promoter or a functional variant thereof. In a more particular embodiment, the CK6 promoter or a functional variant thereof is located upstream the 5′ end of the Acta1 promoter or a functional variant thereof.

In a particular embodiment, the nucleic acid molecule comprises, in this order from 5′ to 3′:
the one or plurality of liver-selective enhancer(s)
a CK6 promoter or a functional variant thereof, and
a spC5-12 promoter or a functional variant thereof; a CK8 promoter or a functional variant thereof; a CK6 promoter or a functional variant thereof; or an Acta1 promoter or a functional variant thereof.

In a particular embodiment, the nucleic acid molecule comprises, in this order from 5′ to 3′:
the one or plurality of liver-selective enhancer(s)
a CK6 promoter or a functional variant thereof, and
a spC5-12 promoter or a functional variant thereof.

In a further particular embodiment, the CK6 promoter consists of the sequence shown in SEQ ID NO: 7 or SEQ ID NO:35, preferably SEQ ID NO:7, or a functional variant having a sequence that is at least 80% identical to SEQ ID NO:7 or SEQ ID NO:35, preferably SEQ ID NO:7, such as at least 85% identical, in particular at least 90% identical, more particularly at least 91%, 92%, 93%, 94%, 95%, 96%, 97%, 98% or even at least 99% identical to SEQ ID NO:7 or SEQ ID NO:35, preferably SEQ ID NO:7.

In a further particular embodiment, the spC5-12 promoter consists of the sequence shown in SEQ ID NO:4, 5 or 6, or a functional variant having a sequence that is at least 80% identical to SEQ ID NO: 4, 5 or 6 such as at least 85% identical, in particular at least 90% identical, more particularly at least 91%, 92%, 93%, 94%, 95%, 96%, 97%, 98% or even at least 99% identical to SEQ ID NO: 4, 5 or 6. Preferably, the spC5-12 promoter consists of the sequence shown in SEQ ID NO:6, or a functional variant having a sequence that is at least 80% identical to SEQ ID NO: 6 such as at least 85% identical, in particular at least 90% identical, more particularly at least 91%, 92%, 93%, 94%, 95%, 96%, 97%, 98% or even at least 99% identical to SEQ ID NO: 6.

In a further particular embodiment, the CK8 promoter consists of the sequence shown in SEQ ID NO: 33 or 34, or a functional variant having a sequence that is at least 80% identical to SEQ ID NO: 33 or 34 such as at least 85% identical, in particular at least 90% identical, more particularly at least 91%, 92%, 93%, 94%, 95%, 96%, 97%, 98% or even at least 99% identical to SEQ ID NO: 33 or 34. Preferably, the CK8 promoter consists of the sequence shown in SEQ ID NO: 33, or a functional variant having a sequence that is at least 80% identical to SEQ ID NO: 33 such as at least 85% identical, in particular at least 90% identical, more particularly at least 91%, 92%, 93%, 94%, 95%, 96%, 97%, 98% or even at least 99% identical to SEQ ID NO: 33.

In a further particular embodiment, the Acta1 promoter consists of the sequence shown in SEQ ID NO: 37, or a functional variant having a sequence that is at least 80% identical to SEQ ID NO: 37 such as at least 85% identical, in particular at least 90% identical, more particularly at least 91%, 92%, 93%, 94%, 95%, 96%, 97%, 98% or even at least 99% identical to SEQ ID NO: 37.

In a further particular embodiment, the nucleic acid molecule comprises one liver-selective enhancer operably linked to the muscle-selective promoters. In another embodiment, the nucleic acid molecule comprises a plurality of liver-selective enhancers operably linked to the muscle-selective promoters. In a particular embodiment, the plurality of liver-selective enhancers comprises at least two liver-selective enhancers. In a further embodiment, the plurality of liver-selective enhancers comprises three liver-selective enhancers. In a specific embodiment, the nucleic acid molecule comprises one, two or three liver-selective enhancers, more particularly three liver-selective enhancers. In a particular embodiment, all the liver-selective enhancers of the plurality of liver-selective enhancers have the same sequence. In a specific embodiment, the plurality of liver-selective enhancers comprises three liver-selective enhancers having the same sequence. In a further particular embodiment, the plurality of liver-selective enhancers comprises three liver-selective enhancers having the same sequence, wherein said sequence comprises of consists of SEQ ID NO:1.

Preferably, the liver-selective enhancer comprises or consists of a sequence selected in the group consisting of SEQ ID NO:1, SEQ ID NO:16, SEQ ID NO:17, SEQ ID NO: 18, SEQ ID NO: 19, SEQ ID NO:20, SEQ ID NO:21, SEQ ID NO:22, SEQ ID NO:23, SEQ ID NO: 24, SEQ ID NO: 25, SEQ ID NO:26, SEQ ID NO:27, SEQ ID NO:28, SEQ ID NO:39, SEQ ID NO:40, SEQ ID NO:41, SEQ ID NO:42 and SEQ ID NO:43 or a functional variant having 80% identity such as at least 85%, in particular at least 90%, more particularly at least 91%, 92%, 93%, 94%, 95%, 96%, 97%, 98% or even at least 99% identity to any one of the sequence selected from SEQ ID NO:1, SEQ ID NO:16, SEQ ID NO: 17, SEQ ID NO: 18, SEQ ID NO: 19, SEQ ID NO: 20, SEQ ID NO:21, SEQ ID NO:22, SEQ ID NO:23, SEQ ID NO:24, SEQ ID NO:25, SEQ ID NO: 26, SEQ ID NO: 27, SEQ ID NO:28, SEQ ID NO:39, SEQ ID NO:40, SEQ ID NO:41, SEQ ID NO:42 and SEQ ID NO:43; or
the plurality of liver-selective enhancers comprises at least one liver-selective enhancer comprising or consisting of a sequence selected in the group consisting of SEQ ID NO: 1, SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO: 27, SEQ ID NO:28, SEQ ID NO:39, SEQ ID NO:40, SEQ ID NO:41, SEQ ID NO:42 and SEQ ID NO:43 or a functional variant having 80% identity such as at least 85%, in particular at least 90%, more particularly at least 91%, 92%, 93%, 94%, 95%, 96%, 97%, 98% or even at least 99% identity to any one of the sequences selected from SEQ ID NO:1, SEQ ID NO: 16, SEQ ID NO:17, SEQ ID NO:18, SEQ ID NO:19, SEQ ID NO:20, SEQ ID NO:21, SEQ ID NO: 22, SEQ ID NO:23, SEQ ID NO:24, SEQ ID NO:25, SEQ ID NO:26, SEQ ID NO: 27, SEQ ID NO: 28, SEQ ID NO:39, SEQ ID NO:40, SEQ ID NO:41, SEQ ID NO:42 and SEQ ID NO: 43.

In a preferred embodiment, the sequence of the liver-selective enhancer comprises or consists of SEQ ID NO: 1, or is a functional variant having a sequence at least 80% identical to SEQ ID NO: 1, such as at least 85% identical, in particular at least 90% identical, more particularly at least 91%, 92%, 93%, 94%, 95%, 96%, 97%, 98% or even at least 99% identical to SEQ ID NO: 1. In a more preferred embodiment, the plurality of liver-selective enhancers consists of three repeats of SEQ ID NO: 1, or three repeats of a functional variant having a sequence at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1.

In a particular embodiment, the nucleic acid molecule of the invention consists of SEQ ID NO: 29, SEQ ID NO:30, SEQ ID NO: 44, SEQ ID NO: 45 or SEQ ID NO:46, or is a functional variant having a sequence at least 80% identical, such as at least 85% identical, in particular at least 90% identical, more particularly at least 91%, 92%, 93%, 94%, 95%, 96%, 97%, 98% or even at least 99% identical to SEQ ID NO:29, SEQ ID NO:30, SEQ ID NO: 44, SEQ ID NO: 45 or SEQ ID NO:46.

The hybrid promoter of the invention may be operably linked to a transgene of interest. Accordingly, the invention further relates to an expression cassette comprising the nucleic acid molecule described herein, operably linked to a transgene of interest.

The invention further relates to a vector comprising the expression cassette described above. In a particular embodiment, the vector is a plasmid vector. In another embodiment, the vector is a viral vector. Representative viral vectors include, without limitation, adenovirus vectors, retrovirus vectors, lentivirus vectors and parvovirus vectors, such as AAV vectors. In a particular embodiment, the viral vector is an AAV vector, such as an AAV vector comprising an AAV8 or AAV9 capsid.

The invention also relates to an isolated recombinant cell comprising the nucleic acid construct or the expression cassette or the vector according to the invention.

The invention further relates to a pharmaceutical composition comprising, in a pharmaceutically acceptable carrier, the vector or the isolated cell of the invention.

Furthermore, the invention also relates to the expression cassette, the vector or the isolated cell disclosed herein, for use as a medicament. In this aspect, the transgene of interest comprised in the expression cassette, the vector or the isolated cell is a therapeutic transgene.

The invention further relates to the expression cassette, the vector or the isolated cell disclosed herein, for use in gene therapy.

In another aspect, the invention relates to the expression cassette, the vector or the isolated cell disclosed herein, for use in the treatment a neuromuscular disorder.

In particular, the neuromuscular disorder may be selected in the group consisting of muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, motor neuron diseases (e.g. amyotrophic lateral sclerosis (ALS), spinal muscular atrophy (Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4)), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphoglycerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenita paramyotonia congenita central core disease nemaline myopathy myotubular myopathy periodic paralysis).

In a further particular embodiment, the disease is Cori disease and the transgene of interest is GDE, such as a truncated form of GDE. In another particular embodiment, the disease is pompe disease.

### LEGEND OF THE FIGURES

FIG. 1. (A) Schematic representation of the enhancer/promoter combinations (P1 to P5).

FIG. 2. Scheme of the in vivo protocol. Mice were injected with an AAV vector encoding murine secreted alkaline phosphatase (mSeAP) under the transcriptional control of P1, P3 or P4 at day 0 and sacrificed 1 month after vector injection.

FIG. 3. AAV vectors bearing the different combinations of enhancer/promoter (P1, P3 or P4) have similar transduction efficacy in vivo. DNA from injected mice tissues was extracted and the transduction efficiency was quantified by qPCR in liver and quadriceps. Data are expressed as vector genome copy number per cell (VGCN).

FIG. 4. mSEAP expression in A) liver, B) muscles (heart, diaphragm, quadriceps and triceps) and C) spinal cord. Data are expressed as a ratio of mSeAP to total protein, quantified in tissues. Statistical analysis was performed by ANOVA. *=p<0.05 vs PBS.

FIG. 5. Scheme of the in vivo protocol. Mice were injected at day 0 with an AAV vector encoding acid-α-glucosidase (GAA) under the transcriptional control of P1-P5 and sacrificed 1 month after vector injection.

FIG. 6. AAV vectors bearing the different combinations of enhancer/promoter (P1 to P5) have similar transduction efficacy in vivo. DNA from injected mice tissues was extracted and the transduction efficiency was quantified by qPCR in heart. Data are expressed as vector genome copy number per cell (VGCN).

FIG. 7. GAA expression A) quantification B) by Western Blot C) in heart. Data are expressed as a ratio of GAA to total protein, quantified in heart. Statistical analysis was performed by ANOVA. *=p<0.05 vs PBS.

FIG. 8. GAA expression A) quantification B) by Western Blot C) in quadriceps. Data are expressed as a ratio of GAA to total protein, quantified in quadriceps. Statistical analysis was performed by ANOVA. *=p<0.05 vs PBS.

FIG. 9. (A) Schematic representation of the enhancer/promoter combinations (P1 to P3). (B) Scheme of the in vivo protocol. Mice were injected at day 0 with an AAV vector encoding acid-α-glucosidase (GAA) under the transcriptional control of P1-P3 and sacrificed 1 month after vector injection.

FIG. 10. (A) Vector genome copy number (VGCN) in heart. GAA expression, quantified by Western Blot, in heart (B-C) and quadriceps (D-E). Data are expressed as a ratio of GAA to vinculin, quantified in heart and quadriceps. Statistical analysis was performed by ANOVA. *=p<0.05 vs PBS.

FIG. 11. (A) Schematic representation of the enhancer/promoter combinations (P1 to P4). (B) Scheme of the in vivo protocol. Mice were injected at day 0 with an AAV vector encoding acid-α-glucosidase (GAA) under the transcriptional control of P1-P4 and sacrificed 1 month after vector injection.

FIG. 12. (A) Vector genome copy number (VGCN) in heart. GAA expression, quantified by Western Blot, in quadriceps (B).

### DETAILED DESCRIPTION

### Definitions

In the context of the present invention, a “transcription regulatory element” is a DNA sequence able to drive or enhance transgene expression in a tissue or cell.

In the context of the present invention, the expression “liver-selective enhancer” includes natural or synthetic liver-selective enhancers. In addition, the expression “muscle-selective promoter” includes natural or synthetic muscle-selective promoters.

According to the present invention tissue selectivity means that a transcription regulatory element preferentially drives (in case of a promoter) or enhances (in case of an enhancer) expression of a gene operably linked to said transcription regulatory element in a given tissue, or set of tissues, as compared to expression in another tissue(s). This definition of “tissue-selectivity” does not exclude the possibility for a tissue-selective transcription regulatory element (such as a muscle-selective promoter) to leak to some extent. By “leak”, “leaking” or declinations thereof, it is meant the possibility for a muscle-selective promoter to drive or increase expression of a transgene operably linked to said promoter into another tissue, although at lower expression levels. For example, a muscle-selective promoter may leak in the liver tissue, meaning that expression drove from this promoter is higher in the muscle tissue than in the liver tissue. Alternatively, the tissue-selective transcription regulatory element may be a “tissue-specific” transcription regulatory element, meaning that this transcription regulatory element not only drives or enhances expression in a given tissue, or set of tissues, in a preferential manner, but also that this regulatory element does not, or does only marginally, drive or enhance expression in other tissues.

The expression “liver-selective enhancer” denotes an enhancer that is particularly effective in enhancing the expression of a transgene in the liver. For example, Chua et al. described a genome-wide in silico method enabling identification of liver-selective transcriptional modules (Chua et al. 2014 Molecular Therapy vol. 22 no. 9, 1605-1613). In particular, the liver-specific enhancer is as defined in Chua et al. In particular, the liver-selective enhancer is a cis-regulatory module associated with highly expressed liver-specific promoters. In particular, the liver-specific enhancer is a cis-regulatory module that contains clusters of evolutionary conserved transcription factor binding sites motifs associated with robust hepatocyte-specific expression.

According to the present invention, a “transgene of interest” refers to a polynucleotide sequence that encodes a RNA or protein product and that may be introduced into a cell for a sought purpose, and is capable of being expressed under appropriate conditions. A transgene of interest may encode a product of interest, for example a therapeutic or diagnostic product of interest. In a particular embodiment, the transgene of interest is a therapeutic transgene, i.e. a transgene that encodes a therapeutic product of interest. A therapeutic transgene is selected and used to lead to a desired therapeutic outcome, in particular for achieving expression of said therapeutic transgene into a cell, tissue or organ into which expression of said therapeutic transgene is needed. Therapy may be achieved by a number of ways, including by expressing a protein into a cell that does not express said protein, by expressing a protein into a cell that expresses a mutated version of the protein, by expressing a protein that is toxic to the target cell into which it is expressed (strategy used, for example, for killing unwanted cells such as cancer cells), by expressing an antisense RNA to induce gene repression or exon skipping, or by expressing a silencing RNA such as a shRNA or micro-RNA whose purpose is to suppress the expression of a protein. The transgene of interest may also encode a nuclease for targeted genome engineering, such as a CRISPR associated protein 9 (Cas9) endonuclease, a meganuclease or a transcription activator-like effector nuclease (TALEN). The transgene of interest may also be a guide RNA or a set of guide RNAs for use with the CRISPR/Cas9 system, or a correcting matrix for use in a targeted genome engineering strategy along with a nuclease as described beforehand. Other transgenes of interest include, without limitation, synthetic long non-coding RNAs (SINEUPs; Carrieri et al., 2012, Nature 491:454-7; Zucchelli et al., 2015, RNA Biol 12 (8): 771-9; Indrieri et al., 2016, Sci Rep 6:27315) and artificial microRNAs. Other specific transgenes of interest useful in the practice of the present invention are described below.

Generally, “operably linked” means that a nucleic acid sequence is placed into a functional relationship with another nucleic acid sequence, so that each nucleic acid sequence can serve its intended function. Two sequences that are operably linked may be directly fused to each other or may be linked via a linker sequence.

The term “functional variant” refers to “functional derivatives”, “fragments”, “analogs”, or “homologs” of a nucleic acid molecule of interest, which retains at least in part the biological activity of said nucleic acid molecule of interest. The functional variant may have an activity which is at least 40%, at least 50%, at least 60%, at least 70%, at least 80%, at least 90%, or at least 95% of the activity of the nucleic acid molecule of interest. For example, a functional variant of a muscle-selective promoter of interest is a variant having an activity at least 40%, at least 50%, at least 60%, at least 70%, at least 80%, at least 90%, or at least 95% of the activity of said muscle-selective promoter, wherein the activity correspond to the ability to enhance the transcription of a particular transgene in muscles.

The term “identical” and declinations thereof refers to the sequence identity between two nucleic acid molecules. When a position in both of the two compared sequences is occupied by the same base, then the molecules are identical at that position. The percent of identity between two sequences is a function of the number of matching positions shared by the two sequences divided by the number of positions compared X 100. For example, if 6 of 10 of the positions in two sequences are matched then the two sequences are 60% identical. Generally, a comparison is made when two sequences are aligned to give maximum identity. Various bioinformatic tools known to the one skilled in the art might be used to align nucleic acid sequences such as BLAST or FASTA.

According to the present invention, the term “treatment” includes curative, alleviation or prophylactic effects. Accordingly, a therapeutic and prophylactic treatment includes amelioration of the symptoms of a disorder or preventing or otherwise reducing the risk of developing a particular disorder. A treatment may be administered to delay, slow or reverse the progression of a disease and/or of one or more of its symptoms. The term “prophylactic” may be considered as reducing the severity or the onset of a particular condition. “Prophylactic” also includes preventing reoccurrence of a particular condition in a patient previously diagnosed with the condition. “Therapeutic” may also refer to the reduction of the severity of an existing condition. By “therapeutic amount” is meant an amount that when administered to a patient suffering from the disorder, is sufficient to cause a qualitative or quantitative reduction in the symptoms of the disorder.

The subject treated in the context of the present invention is an animal, in particular a mammal, more particularly a human subject. In a particular embodiment, said mammal may be an infant or adult subject, such as a human infant or human adult described herein.

By “cell of therapeutic interest” or “tissue of therapeutic interest”, it is meant herein a main cell or tissue where expression of the therapeutic transgene will be useful for the treatment of a disorder. In the present invention, the tissue of interest is the muscle tissue and/or CNS tissue.

Unless otherwise defined, all technical and scientific terms used herein have the same meaning as commonly understood by one of ordinary skill in the art to which this present application belongs.

### Hybrid Promoters

The present inventors have designed a combination of transcription regulatory elements, also referred to herein as “hybrid promoters”, for increasing gene therapy efficacy in muscle and CNS while reducing targeting in the liver and complying with the size constraint of gene therapy vectors, such as the size constraint of AAV vectors.

The nucleic acid molecule of the invention comprises the following transcription regulatory elements, operably linked to each other: one or a plurality of liver-selective enhancer(s) and two muscle-specific promoters.

In a particular embodiment, the nucleic acid molecule of the invention comprises the following transcription regulatory elements, operably linked to each other:
(i) one or a plurality of liver-selective enhancer(s);
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a CK6 promoter, or a functional variant thereof; the second muscle-selective promoter being more preferably a spC5-12 promoter or a functional variant thereof.

The liver-selective enhancer or the plurality of liver-selective enhancer(s) may be selected from liver-selective enhancers known to those skilled in the art. In a particular embodiment, the nucleic acid molecule of the invention comprises one, and only one, liver-selective enhancer. In this embodiment, the size of the liver-selective enhancer may be from 10 to 500 nucleotides, such as from 10 to 175 nucleotides, in particular from 40 to 100 nucleotides, in particular from 50 to 80 nucleotides, more particularly from 70 to 75 nucleotides. In another embodiment, where a plurality of liver-selective enhancers is implemented, the size of the combination of the plurality of liver-selective enhancers may be from 10 to 500 nucleotides, such as from 40 to 400 nucleotides, in particular from 70 to 250 nucleotides. In a preferred embodiment, the size of the sequence corresponding to the liver-selective enhancer or to the plurality of liver-selective enhancers has a length from 50 to 450 pb. In a particular embodiment, the size of the sequence corresponding to the liver-selective enhancer or to the plurality of liver-selective enhancers has a length of at least 50 pb, such as at least 100pb, at least 150 pb, at least 200 pb or at least 250 pb. In a particular embodiment, the liver-selective enhancer is a naturally occurring enhancer located in cis of a gene expressed selectively in hepatocytes. In a further particular embodiment, the liver-selective enhancer may be an artificial liver-selective enhancer.

Illustrative artificial liver-selective enhancers useful in the practice of the present invention include, without limitation, those disclosed in Chuah et al., Molecule Therapy, 2014, vol. 22, no. 9, p. 1605, in particular from HS-CRM1 (SEQ ID NO:16), HS-CRM2 (SEQ ID NO:17), HS-CRM3 (SEQ ID NO:18), HS-CRM4 (SEQ ID NO:19), HS-CRM5 (SEQ ID NO:20), HS-CRM6 (SEQ ID NO:21), HS-CRM7 (SEQ ID NO:22), HS-CRM8 (SEQ ID NO:1), HS-CRM9 (SEQ ID NO:23), HS-CRM10 (SEQ ID NO:24), HS-CRM11 (SEQ ID NO:25), HS-CRM12 (SEQ ID NO:26), HS-CRM13 (SEQ ID NO:27) and HS-CRM14 (SEQ ID NO:28). In a particular embodiment, the liver-selective enhancer may be selected in the group consisting of HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14. In a further particular embodiment, the liver-selective enhancer may be selected in the group consisting of HS-CRM2, HS-CRM7, HS-CRM8, HS-CRM11, HS-CRM13 and HS-CRM14.

Other illustrative liver-selective enhancers useful in the practice of the present invention include the Apolipoprotein E enhancer (ApoE-enhancer sequence shown in SEQ ID NO:39).

In a particular embodiment, the liver-selective enhancer is the Apo-E enhancer consisting of SEQ ID NO:39, or a functional variant of SEQ ID NO:39 having a liver-selective enhancer activity. In another embodiment, the liver-selective enhancer is a functional variant of the Apo-E enhancer that is at least 80% identical to SEQ ID NO:39, such as at least 85% identical, in particular at least 90% identical, more particularly at least 91%, 92%, 93%, 94%, 95%, 96%, 97%, 98% or even at least 99% identical to SEQ ID NO:39, wherein said functional variant has a liver-selective enhancer activity.

Other illustrative liver-selective enhancers useful in the practice of the present invention include the enhancer A3 (SEQ ID NO:40), the enhancer F (SEQ ID NO:41), the enhancer S1 (SEQ ID NO: 42) and the enhancer S2 (SEQ ID NO:43), as described in WO2009/130208 (cf. table III on page 30 of WO2009/130208).

In a particular embodiment, the liver-selective enhancer is the enhancer A3 which regulates expression of the ApoH gene (genomic location sequence: chr17: 61597650-61598200). In a particular embodiment, the liver-selective enhancer is the enhancer A3 consisting of SEQ ID NO: 40, or a functional variant of SEQ ID NO:40 having a liver-selective enhancer activity. In another embodiment, the liver-selective enhancer is a functional variant of the enhancer A3 that is at least 80% identical to SEQ ID NO:40, such as at least 85% identical, in particular at least 90% identical, more particularly at least 91%, 92%, 93%, 94%, 95%, 96%, 97%, 98% or even at least 99% identical to SEQ ID NO:40, wherein said functional variant has a liver-selective enhancer activity.

In a particular embodiment, the liver-selective enhancer is the enhancer F which regulates expression of the FGA gene (genomic location sequence: chr4: 155869502-155869575). In a particular embodiment, the liver-selective enhancer is the enhancer F consisting of SEQ ID NO: 41, or a functional variant of SEQ ID NO:41 having a liver-selective enhancer activity. In another embodiment, the liver-selective enhancer is a functional variant of the enhancer F that is at least 80% identical to SEQ ID NO:41, such as at least 85% identical, in particular at least 90% identical, more particularly at least 91%, 92%, 93%, 94%, 95%, 96%, 97%, 98% or even at least 99% identical to SEQ ID NO:41, wherein said functional variant has a liver-selective enhancer activity.

In a particular embodiment, the liver-selective enhancer is the enhancer S1 which regulates expression of the SERPINA1 gene (genomic location sequence: chr14: 93891375-93891462). In a particular embodiment, the liver-selective enhancer is the enhancer S1 consisting of SEQ ID NO: 42, or a functional variant of SEQ ID NO:42 having a liver-selective enhancer activity. In another embodiment, the liver-selective enhancer is a functional variant of the enhancer S1 that is at least 80% identical to SEQ ID NO:42, such as at least 85% identical, in particular at least 90% identical, more particularly at least 91%, 92%, 93%, 94%, 95%, 96%, 97%, 98% or even at least 99% identical to SEQ ID NO:42, wherein said functional variant has a liver-selective enhancer activity.

In a particular embodiment, the liver-selective enhancer is the enhancer S2 which regulates expression of the SERPINA1 gene (genomic location sequence: chr14: 93897160-93897200). In a particular embodiment, the liver-selective enhancer is the enhancer S2 consisting of SEQ ID NO: 43, or a functional variant of SEQ ID NO:43 having a liver-selective enhancer activity. In another embodiment, the liver-selective enhancer is a functional variant of the enhancer S1 that is at least 80% identical to SEQ ID NO:43, such as at least 85% identical, in particular at least 90% identical, more particularly at least 91%, 92%, 93%, 94%, 95%, 96%, 97%, 98% or even at least 99% identical to SEQ ID NO:43, wherein said functional variant has a liver-selective enhancer activity.

In a particular embodiment, the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14.

In a particular embodiment, the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), and HS-CRM8.

In a particular embodiment, the liver-selective enhancer is selected in the group consisting of ApoE enhancer and HS-CRM8.

In a particular embodiment, the liver-selective enhancer is HS-CRM8.

In a particular embodiment, the liver-selective enhancer is the HS-CRM8 enhancer consisting of SEQ ID NO:1, or a functional variant of SEQ ID NO:1 having a liver-selective enhancer activity. In another embodiment, the liver-selective enhancer is a functional variant of the HS-CRM8 enhancer that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional variant has a liver-selective enhancer activity. In case of a plurality of liver-selective enhancers, said enhancers may be fused directly, or separated by a linker (same or different linkers). A direct fusion means that the first nucleotide of an enhancer immediately follows the last nucleotide of an upstream enhancer. In case of a link via a linker, a nucleotide sequence is present between the last nucleotide of an upstream enhancer and the first nucleotide of the following downstream enhancer. For example, the length of the linker may be comprised between 1 and 50 nucleotides, such as from 1 to 40 nucleotides, such as from 1 to 30 nucleotides, such as from 1 to 20 nucleotides, such as from 1 to 10 nucleotides. In the present invention, the design of the nucleic molecule may take into account the size constraints mentioned above and therefore, such linker(s), if any, are preferably short. Representative short linkers comprise nucleic acid sequences consisting of less than 15 nucleotides, in particular of less than 14, 13, 12, 11, 10, 9, 8, 7, 6, 5, 4, 3 or less than 2 nucleotides, such as a linker of 1 nucleotide. In a particular embodiment, the linker is a restriction site. In a particular embodiment, the linker is AAGCTT.

In a particular embodiment, the nucleic acid molecule comprises a plurality of liver-selective enhancers, i.e. at least two liver-selective enhancers or a least three liver-selective enhancers. The number of liver-selective enhancers may be determined by the skilled person, depending on the size of the transgene whose expression is controlled by the nucleic acid molecule of the invention. In a particular embodiment, the plurality of liver-selective enhancers comprises at least two liver-selective enhancers and at most ten liver-selective enhancers. In a particular embodiment, the plurality of liver-selective enhancers comprises at least two liver-selective enhancers and at most six liver-selective enhancers. In yet another embodiment, the plurality of liver-selective enhancers comprises two liver-selective enhancers. In a further embodiment, the plurality of liver-selective enhancers comprises three liver-selective enhancers. In a further embodiment, the plurality of liver-selective enhancers comprises four liver-selective enhancers. In yet another embodiment, the plurality of liver-selective enhancers comprises five liver-selective enhancers. In a specific embodiment, the nucleic acid molecule comprises one, two or three liver-selective enhancers, more particularly one or three liver-selective enhancers. In a particular embodiment, all the liver-selective enhancers of the plurality of liver-selective enhancers have the same sequence. In a particular embodiment, at least two of the liver-selective enhancers of the plurality of liver-selective enhancers have a different sequence.

In a particular embodiment, the nucleic acid molecule comprises one, two, three, four or five repeats of the S1 enhancer consisting of SEQ ID NO:42, or a functional variant of SEQ ID NO: 42 having a liver-selective enhancer activity as described above.

In a particular embodiment, the nucleic acid molecule comprises one, two, three, four or five repeats of the S2 enhancer consisting of SEQ ID NO:43, or a functional variant of SEQ ID NO: 43 having a liver-selective enhancer activity as described above.

In a particular embodiment, the nucleic acid molecule comprises one, two, three, four or five repeats of the enhancer F consisting of SEQ ID NO:41, or a functional variant of SEQ ID NO:41 having a liver-selective enhancer activity as described above. In a particular embodiment, the nucleic acid molecule comprises three repeats of the enhancer F consisting of SEQ ID NO:41, or a functional variant of SEQ ID NO:41 having a liver-selective enhancer activity as described above.

In a specific embodiment, all the liver-selective enhancers of the plurality of liver-selective enhancers have the same sequence. In a preferred embodiment, all the liver-selective enhancers of the plurality of liver-selective enhancers have the same sequence, which is the sequence of SEQ ID NO:1, or a functional variant of SEQ ID NO:1 having a liver-selective enhancer activity, as described above.

In a particular embodiment, the nucleic acid molecule comprises one, two, three, four or five repeats of the HS-CRM8 enhancer consisting of SEQ ID NO:1, or a functional variant of SEQ ID NO: 1 having a liver-selective enhancer activity as described above. In a particular embodiment, the nucleic acid molecule of the invention comprises three repeats of the HS-CRM8 enhancer consisting of SEQ ID NO:1, or a functional variant of SEQ ID NO: 1 having a liver-selective enhancer activity. In another embodiment, the nucleic acid molecule of the invention comprises three repeats of a functional variant of the HS-CRM8 enhancer that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional variant has a liver-selective enhancer activity. Said liver-selective enhancer activity may be determined as described in Chua et al. (Chua et al. 2014 Molecular Therapy vol. 22 no. 9, 1605-1613).

In a particular embodiment, the sequence corresponding to the plurality of liver-selective enhancers is SEQ ID NO:2 or SEQ ID NO:3, or a functional variant that is at least 80% identical to SEQ ID NO:2 or 3, such as at least 85% identical, in particular at least 90% identical, more particularly at least 91%, 92%, 93%, 94%, 95%, 96%, 97%, 98% or even at least 99% identical to SEQ ID NO:2 or 3. SEQ ID NO:2 and SEQ ID NO:3 comprise three repeats of the HS-CRM8 enhancer of SEQ ID NO:1.

In addition, but optionally, the nucleic acid molecule may comprise a further liver-selective enhancer or a further plurality of liver-selective enhancer(s). According to this embodiment, the nucleic acid molecule may comprise in this order from 5′ to 3′:
a first liver-selective enhancer or a first plurality of liver-selective enhancers, such as two or three liver-selective enhancers;
the first muscle-selective promoter as defined above (i.e. a CK6 promoter or functional variant thereof);
a second liver-selective enhancer or a second plurality of liver-selective enhancers, such as two or three liver-selective enhancers; and
the second muscle-selective promoter as defined above.

According to another variant of this embodiment, the nucleic acid molecule may comprise in this order from 5′ to 3′:
a first liver-selective enhancer or a first plurality of liver-selective enhancers, such as two or three liver-selective enhancers;
the second muscle-selective promoter, as defined above (i.e. a muscle-selective promoter selected in the group consisting of: a spC5-12 promoter, CK6 promoter, CK8 promoter, MCK promoter, Acta1 promoter, desmin promoter, and functional variants thereof, preferably spC5-12 or a functional variant thereof);
a second liver-selective enhancer or a second plurality of liver-selective enhancers, such as two or three liver-selective enhancers; and
the first muscle-selective promoter, as defined above (i.e. a CK6 promoter or functional variant thereof).

According to this embodiment, the first liver-selective enhancer or plurality of liver-selective enhancers and the second liver-selective enhancer or plurality of liver-selective enhancers may be any of the liver-selective enhancers or plurality of liver-selective enhancers as described above.

In a particular embodiment, the nucleic acid molecule may comprise in this order from 5′ to 3′:
a first liver-selective enhancer or a first plurality of liver-selective enhancers, wherein the liver-selective enhancer is selected from ApoE enhancer (SEQ ID NO:39), enhancer A3 (SEQ ID NO:40), enhancer F (SEQ ID NO:41), enhancer S1 (SEQ ID NO: 42), enhancer S2 (SEQ ID NO:43), HS-CRM1 (SEQ ID NO:16), HS-CRM2 (SEQ ID NO: 17), HS-CRM3 (SEQ ID NO:18), HS-CRM4 (SEQ ID NO:19), HS-CRM5 (SEQ ID NO: 20), HS-CRM6 (SEQ ID NO:21), HS-CRM7 (SEQ ID NO:22), HS-CRM8 (SEQ ID NO:1), HS-CRM9 (SEQ ID NO:23), HS-CRM10 (SEQ ID NO:24), HS-CRM11 (SEQ ID NO:25), HS-CRM12 (SEQ ID NO:26), HS-CRM13 (SEQ ID NO:27) and HS-CRM14 (SEQ ID NO:28), in particular HS-CRM8 (SEQ ID NO:1);
a muscle-selective promoter, which is a CK6 promoter or functional variant thereof;
a second liver-selective enhancer or a second plurality of liver-selective enhancers, wherein the liver-selective enhancer is selected from ApoE enhancer (SEQ ID NO:39), enhancer A3 (SEQ ID NO:40), enhancer F (SEQ ID NO:41), enhancer S1 (SEQ ID NO: 42), enhancer S2 (SEQ ID NO:43), HS-CRM1 (SEQ ID NO:16), HS-CRM2 (SEQ ID NO: 17), HS-CRM3 (SEQ ID NO:18), HS-CRM4 (SEQ ID NO:19), HS-CRM5 (SEQ ID NO: 20), HS-CRM6 (SEQ ID NO:21), HS-CRM7 (SEQ ID NO:22), HS-CRM8 (SEQ ID NO:1), HS-CRM9 (SEQ ID NO:23), HS-CRM10 (SEQ ID NO:24), HS-CRM11 (SEQ ID NO:25), HS-CRM12 (SEQ ID NO:26), HS-CRM13 (SEQ ID NO:27) and HS-CRM14 (SEQ ID NO:28), in particular HS-CRM8 (SEQ ID NO:1); and
a muscle-selective promoter selected in the group consisting of: a spC5-12 promoter, CK6 promoter, CK8 promoter, MCK promoter, Acta1 promoter, desmin promoter, and functional variants thereof, preferably spC5-12 or a functional variant thereof.

According to another variant of this embodiment, the nucleic acid molecule may comprise in this order from 5′ to 3′:
a first liver-selective enhancer or a first plurality of liver-selective enhancers, wherein the liver-selective enhancer is selected from ApoE enhancer (SEQ ID NO:39), enhancer A3 (SEQ ID NO:40), enhancer F (SEQ ID NO:41), enhancer S1 (SEQ ID NO: 42), enhancer S2 (SEQ ID NO:43), HS-CRM1 (SEQ ID NO:16), HS-CRM2 (SEQ ID NO: 17), HS-CRM3 (SEQ ID NO:18), HS-CRM4 (SEQ ID NO:19), HS-CRM5 (SEQ ID NO: 20), HS-CRM6 (SEQ ID NO:21), HS-CRM7 (SEQ ID NO:22), HS-CRM8 (SEQ ID NO:1), HS-CRM9 (SEQ ID NO:23), HS-CRM10 (SEQ ID NO:24), HS-CRM11 (SEQ ID NO:25), HS-CRM12 (SEQ ID NO:26), HS-CRM13 (SEQ ID NO:27) and HS-CRM14 (SEQ ID NO:28), in particular HS-CRM8 (SEQ ID NO:1);
a muscle-selective promoter selected in the group consisting of: a spC5-12 promoter, CK6 promoter, CK8 promoter, MCK promoter, Acta1 promoter, desmin promoter, and functional variants thereof, preferably spC5-12 or a functional variant thereof;
a second liver-selective enhancer or a second plurality of liver-selective enhancers, wherein the liver-selective enhancer is selected from ApoE enhancer (SEQ ID NO:39), enhancer A3 (SEQ ID NO:40), enhancer F (SEQ ID NO:41), enhancer S1 (SEQ ID NO: 42), enhancer S2 (SEQ ID NO:43), HS-CRM1 (SEQ ID NO:16), HS-CRM2 (SEQ ID NO: 17), HS-CRM3 (SEQ ID NO:18), HS-CRM4 (SEQ ID NO:19), HS-CRM5 (SEQ ID NO: 20), HS-CRM6 (SEQ ID NO:21), HS-CRM7 (SEQ ID NO:22), HS-CRM8 (SEQ ID NO:1), HS-CRM9 (SEQ ID NO:23), HS-CRM10 (SEQ ID NO:24), HS-CRM11 (SEQ ID NO:25), HS-CRM12 (SEQ ID NO:26), HS-CRM13 (SEQ ID NO:27) and HS-CRM14 (SEQ ID NO:28), in particular HS-CRM8 (SEQ ID NO:1); and
a muscle-selective promoter, which is a CK6 promoter or a functional variant thereof.

In a particular embodiment, the nucleic acid molecule of the invention comprises the following transcription regulatory elements, operably linked to each other:
(i) the Apo-E enhancer of SEQ ID NO:39 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter, a CK6 promoter or a functional variant thereof; the second muscle-selective promoter being more preferably a spC5-12 promoter or a functional variant thereof.

In a particular embodiment, the nucleic acid molecule of the invention comprises the following transcription regulatory elements, operably linked to each other, in this order from 5′ to 3′:
(i) the Apo-E enhancer of SEQ ID NO:39 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter, a CK6 promoter or a functional variant thereof; the second muscle-selective promoter being more preferably a spC5-12 promoter or a functional variant thereof.

In a particular embodiment, the nucleic acid molecule of the invention comprises the following transcription regulatory elements, operably linked to each other, in this order from 5′ to 3′:
(i) the Apo-E enhancer of SEQ ID NO:39 or a functional variant thereof;
(ii) a first muscle-selective promoter, which is a CK6 promoter of SEQ ID NO: 7 or a functional variant thereof; and
(iii) a second muscle-selective promoter, which is the spC5-12 promoter of SEQ ID NO: 4, 5 or 6, in particular the sequence shown in SEQ ID NO:6 or a functional variant thereof.

In another particular embodiment, the nucleic acid molecule of the invention comprises the following transcription regulatory elements, operably linked to each other, preferably in this order from 5′ to 3′:
(i) the enhancer A3 of SEQ ID NO: 40 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer A3 of SEQ ID NO: 40 or a functional variant thereof;
(ii) a first muscle-selective promoter, which is a CK6 promoter of SEQ ID NO: 7 or a functional variant thereof; and
(iii) a second muscle-selective promoter, which is the spC5-12 promoter of SEQ ID NO: 4, 5 or 6, in particular the sequence shown in SEQ ID NO:6 or a functional variant thereof.

In another particular embodiment, the nucleic acid molecule of the invention comprises the following transcription regulatory elements, operably linked to each other, preferably in this order from 5′ to 3′:
(i) the enhancer F of SEQ ID NO: 41 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer F of SEQ ID NO: 41 or a functional variant thereof;
(ii) a first muscle-selective promoter, which is a CK6 promoter of SEQ ID NO: 7 or a functional variant thereof; and
(iii) a second muscle-selective promoter, which is the spC5-12 promoter of SEQ ID NO: 4, 5 or 6, in particular the sequence shown in SEQ ID NO:6 or a functional variant thereof.

In another particular embodiment, the nucleic acid molecule of the invention comprises the following transcription regulatory elements, operably linked to each other, preferably in this order from 5′ to 3′:
a first plurality of liver-selective enhancers consisting of one, two, three, four or five repeats of the enhancer F of SEQ ID NO: 41 or a functional variant thereof having a liver-selective enhancer activity as described above;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof, preferably spC5-12 promoter or a functional variant thereof.

In another particular embodiment, the nucleic acid molecule of the invention comprises the following transcription regulatory elements, operably linked to each other, preferably in this order from 5′ to 3′:
a first plurality of liver-selective enhancers consisting of three repeats of the enhancer F of SEQ ID NO: 41 or a functional variant thereof having a liver-selective enhancer activity as described above;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof, preferably spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer S1 of SEQ ID NO: 42 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, Acta1 promoter, MCK promoter, desmin promoter, and functional variants thereof; the second muscle-selective promoter being preferably a spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer S1 of SEQ ID NO: 42 or a functional variant thereof;
(ii) a first muscle-selective promoter, which is a CK6 promoter of SEQ ID NO: 7 or a functional variant thereof; and
(iii) a second muscle-selective promoter, which is the spC5-12 promoter of SEQ ID NO: 4, 5 or 6, in particular the sequence shown in SEQ ID NO:6 or a functional variant thereof.

In another particular embodiment, the nucleic acid molecule of the invention comprises the following transcription regulatory elements, operably linked to each other, preferably in this order from 5′ to 3′:
a first plurality of liver-selective enhancers consisting of one, two, three, four or five repeats of the enhancer S1 of SEQ ID NO: 42 or a functional variant thereof having a liver-selective enhancer activity as described above;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof, preferably spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer S2 of SEQ ID NO: 43 or a functional variant thereof;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof.

In another particular embodiment, the nucleic acid molecule of the invention comprises the following transcription regulatory elements, operably linked to each other, preferably in this order from 5′ to 3′:
(i) the enhancer S2 of SEQ ID NO: 43 or a functional variant thereof;
(ii) a first muscle-selective promoter, which is a CK6 promoter of SEQ ID NO: 7 or a functional variant thereof; and
(iii) a second muscle-selective promoter, which is the spC5-12 promoter of SEQ ID NO: 4, 5 or 6, in particular the sequence shown in SEQ ID NO:6 or a functional variant thereof.

In another particular embodiment, the nucleic acid molecule of the invention comprises the following transcription regulatory elements, operably linked to each other, preferably in this order from 5′ to 3′:
a first plurality of liver-selective enhancers consisting of one, two, three, four or five repeats of the enhancer S2 of SEQ ID NO: 43 or a functional variant thereof having a liver-selective enhancer activity as described above;
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, MCK promoter, Acta1 promoter, desmin promoter, and functional variants thereof; the second muscle-selective promoter being preferably a spC5-12 promoter or a functional variant thereof, preferably spC5-12 promoter or a functional variant thereof.

In a particular embodiment, the nucleic acid molecule may comprise in this order from 5′ to 3′:
a first plurality of liver-selective enhancers consisting of one, two, three, four or five repeats of the HS-CRM8 enhancer as shown in SEQ ID NO:1, or a functional variant thereof having a liver-selective enhancer activity as described above;
a first muscle-selective promoter, which is a CK6 promoter or a functional variant thereof;
a second plurality of liver-selective enhancers consisting of one, two, three, four or five repeats of the HS-CRM8 enhancer as shown in SEQ ID NO:1, or a functional variant thereof having a liver-selective enhancer activity as described above; and
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

In a particular embodiment, the nucleic acid molecule may comprise in this order from 5′ to 3′:
a first plurality of liver-selective enhancers consisting of one, two, three, four or five repeats of the HS-CRM8 enhancer as shown in SEQ ID NO:1, or a functional variant thereof having a liver-selective enhancer activity as described above;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof;
a second plurality of liver-selective enhancers consisting of one, two, three, four or five repeats of the HS-CRM8 enhancer as shown in SEQ ID NO:1, or a functional variant thereof having a liver-selective enhancer activity as described above; and
a muscle-selective promoter, which is a CK6 promoter or a functional variant thereof.

In a further particular embodiment, the nucleic acid molecule may comprise in this order from 5′ to 3′:
a first plurality of liver-selective enhancers consisting of three repeats of the HS-CRM8 enhancer as shown in SEQ ID NO:1, or a functional variant thereof having a liver-selective enhancer activity as described above;
a muscle-selective promoter, which is a CK6 promoter or a functional variant thereof;
a second plurality of liver-selective enhancers consisting of three repeats of the HS-CRM8 enhancer as shown in SEQ ID NO:1, or a functional variant thereof having a liver-selective enhancer activity as described above; and
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

In a particular embodiment, the nucleic acid molecule may comprise in this order from 5′ to 3′:
a first plurality of liver-selective enhancers consisting of three repeats of the HS-CRM8 enhancer as shown in SEQ ID NO:1, or a functional variant thereof having a liver-selective enhancer activity as described above;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof;
a second plurality of liver-selective enhancers consisting of three repeats of the HS-CRM8 enhancer as shown in SEQ ID NO:1, or a functional variant thereof having a liver-selective enhancer activity as described above; and
a muscle-selective promoter, which is a CK6 promoter or a functional variant thereof.

In a particular embodiment, the sequence of the CK6 promoter or the functional variant thereof is selected from:
a sequence that consists of the sequence shown in SEQ ID NO:7;
a functional fragment having at most 10 extra nucleotides or at most 10 missing nucleotides when compared to SEQ ID NO:7, wherein said fragment has a muscle-selective promoter activity;
a sequence which is a functional variant of the sequence shown in SEQ ID NO:7, that consists of a sequence that is at least 80% identical to SEQ ID NO:7, such as at least 85% identical, in particular at least 90% identical, more particularly at least 91%, 92%, 93%, 94%, 95%, 96%, 97%, 98% or even at least 99% identical to SEQ ID NO:7.

By “functional variant” of CK6 is meant a variant which retains at least in part the biological activity of the CK6 promoter. The functional variant may have an activity which is at least 40%, at least 50%, at least 60%, at least 70%, at least 80%, at least 90%, or at least 95% of the activity of the CK6 promoter. For example, a functional variant of a CK6 promoter is a variant having an activity at least 40%, at least 50%, at least 60%, at least 70%, at least 80%, at least 90%, or at least 95% of the activity of CK6 promoter, wherein the activity corresponds to the ability of CK6 to enhance the transcription of a particular transgene in muscles. In a particular embodiment, the sequence of the CK6 promoter consists of SEQ ID NO:7 or SEQ ID NO:35, or a functional variant thereof having a sequence that is at least 80% identical to SEQ ID NO:7 or SEQ ID NO:35, such as at least 85% identical, in particular at least 90% identical, more particularly at least 91%, 92%, 93%, 94%, 95%, 96%, 97%, 98% or even at least 99% identical to SEQ ID NO:7 or SEQ ID NO:35.

In a particular embodiment, the second muscle-selective promoter is a synthetic promoter C5.12 (spC5.12, alternatively referred to herein as “C5.12”), such as a spC5.12 shown in SEQ ID NO: 4, 5 or 6 or the spC5.12 promoter disclosed in Wang et al., Gene Therapy volume 15, pages 1489-1499 (2008). In a particular embodiment, the sequence of the spC5-12 promoter or the functional variant thereof is selected from:
a sequence that consists of the sequence shown in SEQ ID NO: 4, 5 or 6, in particular the sequence shown in SEQ ID NO:6;
a functional fragment having at most 10 extra nucleotides or at most 10 missing nucleotides when compared to SEQ ID NO: 4, 5 or 6, in particular when compared to SEQ ID NO:6, wherein said fragment has a muscle-selective promoter activity;
a sequence which is a functional variant of the sequence shown in SEQ ID NO: 4, 5 or 6, in particular the sequence shown in SEQ ID NO:6, that consists of a sequence that is at least 80% identical to any one of SEQ ID NO: 4, 5 or 6, in particular to SEQ ID NO:6, such as at least 85% identical, in particular at least 90% identical, more particularly at least 91%, 92%, 93%, 94%, 95%, 96%, 97%, 98% or even at least 99% identical to any one of SEQ ID NO: 4, 5 or 6, in particular to SEQ ID NO:6.

By “functional variant” of spC5-12 is meant a variant, which retains at least in part the biological activity of the spC5-12 promoter. The functional variant may have an activity which is at least 40%, at least 50%, at least 60%, at least 70%, at least 80%, at least 90%, or at least 95% of the activity of the spC5-12 promoter. For example, a functional variant of a spC5-12 promoter is a variant having an activity at least 40%, at least 50%, at least 60%, at least 70%, at least 80%, at least 90%, or at least 95% of the activity of spC5-12 promoter, wherein the activity corresponds to the ability of spC5-12 to enhance the transcription of a particular transgene in muscles.

In a particular embodiment, the second muscle-selective promoter is a CK8 promoter. In a particular embodiment, the sequence of the CK8 promoter or the functional variant thereof is selected from:
a sequence that consists of the sequence shown in SEQ ID NO:33;
a functional fragment having at most 10 extra nucleotides or at most 10 missing nucleotides when compared to SEQ ID NO:33, wherein said fragment has a muscle-selective promoter activity;
a sequence which is a functional variant of the sequence shown in SEQ ID NO:33, that consists of a sequence that is at least 80% identical to SEQ ID NO:33, such as at least 85% identical, in particular at least 90% identical, more particularly at least 91%, 92%, 93%, 94%, 95%, 96%, 97%, 98% or even at least 99% identical to SEQ ID NO:33.

By “functional variant” of CK8 is meant a variant which retains at least in part the biological activity of the CK8 promoter. The functional variant may have an activity which is at least 40%, at least 50%, at least 60%, at least 70%, at least 80%, at least 90%, or at least 95% of the activity of the CK8 promoter. For example, a functional variant of a CK8 promoter is a variant having an activity at least 40%, at least 50%, at least 60%, at least 70%, at least 80%, at least 90%, or at least 95% of the activity of CK8 promoter, wherein the activity corresponds to the ability of CK8 to enhance the transcription of a particular transgene in muscles. In a particular embodiment, the sequence of the CK8 promoter consists of SEQ ID NO:33 or SEQ ID NO:34 or a functional variant thereof having a sequence that is at least 80% identical to SEQ ID NO:33 or SEQ ID NO:34, such as at least 85% identical, in particular at least 90% identical, more particularly at least 91%, 92%, 93%, 94%, 95%, 96%, 97%, 98% or even at least 99% identical to SEQ ID NO:33 or SEQ ID NO:34.

In a particular embodiment, the second muscle-selective promoter is a MCK promoter. In a particular embodiment, the sequence of the MCK promoter or the functional variant thereof is selected from:
a sequence that consists of the sequence shown in SEQ ID NO:36;
a functional fragment having at most 10 extra nucleotides or at most 10 missing nucleotides when compared to SEQ ID NO:36, wherein said fragment has a muscle-selective promoter activity;
a sequence which is a functional variant of the sequence shown in SEQ ID NO:36, that consists of a sequence that is at least 80% identical to SEQ ID NO:36, such as at least 85% identical, in particular at least 90% identical, more particularly at least 91%, 92%, 93%, 94%, 95%, 96%, 97%, 98% or even at least 99% identical to SEQ ID NO:36.

By “functional variant” of MCK is meant a variant, which retains at least in part the biological activity of the MCK promoter. The functional variant may have an activity which is at least 40%, at least 50%, at least 60%, at least 70%, at least 80%, at least 90%, or at least 95% of the activity of the MCK promoter. For example, a functional variant of a MCK promoter is a variant having an activity at least 40%, at least 50%, at least 60%, at least 70%, at least 80%, at least 90%, or at least 95% of the activity of MCK promoter, wherein the activity corresponds to the ability of MCK to enhance the transcription of a particular transgene in muscles.

In a particular embodiment, the second muscle-selective promoter is a Acta1 promoter. In a particular embodiment, the sequence of the Acta1 promoter or the functional variant thereof is selected from:
a sequence that consists of the sequence shown in SEQ ID NO:37;
a functional fragment having at most 10 extra nucleotides or at most 10 missing nucleotides when compared to SEQ ID NO:37, wherein said fragment has a muscle-selective promoter activity;
a sequence which is a functional variant of the sequence shown in SEQ ID NO:37, that consists of a sequence that is at least 80% identical to SEQ ID NO:37, such as at least 85% identical, in particular at least 90% identical, more particularly at least 91%, 92%, 93%, 94%, 95%, 96%, 97%, 98% or even at least 99% identical to SEQ ID NO:37.

By “functional variant” of Acta1 is meant a variant which retains at least in part the biological activity of the Acta1 promoter. The functional variant may have an activity which is at least 40%, at least 50%, at least 60%, at least 70%, at least 80%, at least 90%, or at least 95% of the activity of the Acta1 promoter. For example, a functional variant of a Acta1 promoter is a variant having an activity at least 40%, at least 50%, at least 60%, at least 70%, at least 80%, at least 90%, or at least 95% of the activity of Acta1 promoter, wherein the activity corresponds to the ability of Acta1 to enhance the transcription of a particular transgene in muscles.

In a particular embodiment, the second muscle-selective promoter is a desmin promoter. In a particular embodiment, the sequence of the desmin promoter or the functional variant thereof is selected from:
a sequence that consists of the sequence shown in SEQ ID NO:38;
a functional fragment having at most 10 extra nucleotides or at most 10 missing nucleotides when compared to SEQ ID NO:38, wherein said fragment has a muscle-selective promoter activity;
a sequence which is a functional variant of the sequence shown in SEQ ID NO:38, that consists of a sequence that is at least 80% identical to SEQ ID NO:38, such as at least 85% identical, in particular at least 90% identical, more particularly at least 91%, 92%, 93%, 94%, 95%, 96%, 97%, 98% or even at least 99% identical to SEQ ID NO:38.

By “functional variant” of desmin is meant a variant which retains at least in part the biological activity of the desmin promoter. The functional variant may have an activity which is at least 40%, at least 50%, at least 60%, at least 70%, at least 80%, at least 90%, or at least 95% of the activity of the desmin promoter. For example, a functional variant of a desmin promoter is a variant having an activity at least 40%, at least 50%, at least 60%, at least 70%, at least 80%, at least 90%, or at least 95% of the activity of desmin promoter, wherein the activity corresponds to the ability of desmin to enhance the transcription of a particular transgene in muscles.

In the context of the present invention, the transcription regulatory elements (i.e. (i) the liver-selective enhancer or the plurality of enhancer(s); (ii) the optional further liver-selective enhancer or plurality of enhancer(s); (iv) the first muscle selective promoter (i.e. CK6 promoter); and (v) the second muscle selective promoter, which is preferably the spC5-12 promoter) introduced into the nucleic acid molecule of the invention may be either fused directly or linked via a linker.

In a particular embodiment, the nucleic acid molecule of the invention comprises (i) one or a plurality of liver-selective enhancer(s) as described above which is linked via a linker, in particular a linker of sequence ACTAGT or CGCGCC to (ii) a CK6 promoter or a functional variant thereof; the CK6 promoter being linked via a linker, in particular a linker of sequence TTAATGACCC (SEQ ID NO:8) or TTCC to (iii) a second promoter, the second promoter being selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

Preferably, the nucleic acid molecule of the invention comprises (i) one or a plurality of liver-selective enhancer(s) as described above which is linked via a linker, in particular a linker of sequence CGCGCC to (ii) a CK6 promoter or a functional variant thereof; the CK6 promoter being linked via a linker, in particular a linker of sequence TTCC to (iii) a second promoter, the second promoter being selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

For example, in case of one liver-selective enhancer fused directly to a CK6 promoter, a direct fusion means that the first nucleotide of the CK6 promoter immediately follows the last nucleotide of the liver-selective enhancer. In addition, in case of a design with a plurality of liver-selective enhancers fused directly to a CK6 promoter, a direct fusion means that the first nucleotide of the CK6 promoter immediately follows the last nucleotide of the most 3′ liver-selective enhancer.

For example, in case of one liver-selective enhancer fused directly to a spC5-12 promoter, a direct fusion means that the first nucleotide of the spC5-12 promoter immediately follows the last nucleotide of the liver-selective enhancer. In addition, in case of a design with a plurality of liver-selective enhancers fused directly to a spC5-12 promoter, a direct fusion means that the first nucleotide of the spC5-12 promoter immediately follows the last nucleotide of the most 3′ liver-selective enhancer.

In case of a link of two transcription regulatory elements via a linker, a nucleotide sequence is present between:
the last nucleotide of the first transcription regulatory element; and
the first nucleotide of the second transcription regulatory element.

For example, in case of a link of a liver-selective enhancer and a CK6 promoter via a linker, a nucleotide sequence is present between:
the last nucleotide of the liver-selective enhancer; and
the first nucleotide of the CK6 promoter.

For example, in case of a link of a liver-selective enhancer and a spC5-12 promoter via a linker, a nucleotide sequence is present between:
the last nucleotide of the liver-selective enhancer; and
the first nucleotide of the spC5-12 promoter.

According to another example, in case of a link of a plurality of liver-selective enhancers and a CK6 promoter via a linker, a nucleotide sequence is present between:
the last nucleotide of the most 3′ liver-selective enhancer; and
the first nucleotide of the CK6 promoter.

According to another example, in case of a link of a plurality of liver-selective enhancers and a spC5-12 promoter via a linker, a nucleotide sequence is present between:
the last nucleotide of the most 3′ liver-selective enhancer; and
the first nucleotide of the spC5-12 promoter.

The length of the linker between the enhancer or the plurality of enhancers and the first promoter may be comprised between 1 and 1500 nucleotides, such as from 1 to 1000 nucleotides (e.g. 101, 300, 500 or 1000 nucleotides), such as from 1 and 500 nucleotides, such as from 1 and 300 nucleotides, such as from 1 and 100 nucleotides, such as from 1 to 50 nucleotides, such as from 1 to 40 nucleotides, such as from 1 to 30 nucleotides, such as from 1 to 20 nucleotides, such as from 1 to 10 nucleotides. In the present invention, the design of the nucleic molecule may take into account the size constraints of the vector, in particular an AAV vector, and therefore such linker(s), if any, is preferably short. Representative short linkers comprise nucleic acid sequences consisting of less than 15 nucleotides, in particular of less than 14, 13, 12, 11, 10, 9, 8, 7, 6, 5, 4, 3 or less than 2 nucleotides, such as a linker of 1 nucleotide.

In a particular embodiment, the nucleic acid molecule of the invention comprises, in particular in this order from 5′ to 3′:
one or a plurality of liver-selective enhancer(s) as described above;
a CK6 promoter or a functional variant thereof; and
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

In another particular embodiment, the nucleic acid molecule of the invention comprises, in particular in this order from 5′ to 3′:
one or a plurality of liver-selective enhancer(s) as described above;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof; and
a CK6 promoter or a functional variant thereof.

In another particular embodiment, the nucleic acid molecule of the invention comprises, in particular in this order from 5′ to 3′:
one or a plurality of liver-selective enhancer(s) as described above;
a muscle-selective promoter selected in the group consisting of: CK6 promoter, CK8 promoter, MCK promoter, Acta1 promoter, desmin promoter, and functional variants thereof; and
a CK6 promoter or a functional variant thereof.

In a particular embodiment, the nucleic acid molecule of the invention comprises, in particular in this order from 5′ to 3′:
one or a plurality of liver-selective enhancer(s) as described above;
a CK6 promoter or a functional variant thereof;
one or a plurality of liver-selective enhancer(s) as described above; and
a muscle-selective promoter selected in the group consisting of: a spC5-12 promoter, CK6 promoter, CK8 promoter, MCK promoter, Acta1 promoter, desmin promoter and functional variants thereof, preferably a spC5-12 promoter or a functional variant thereof.

According to a particular variant of this embodiment, the nucleic acid molecule of the invention consists of the sequence shown in SEQ ID NO:31, or a functional variant thereof having a sequence at least 80% identical to SEQ ID NO:31, such as at least 85%, 90%, 91%, 92%, 93%, 94%, 95%, 96%, 97%, 98% or even at least 99% identical to SEQ ID NO:31.

In another particular embodiment, the nucleic acid molecule of the invention comprises, in particular in this order from 5′ to 3′:
one or a plurality of liver-selective enhancer(s) as described above;
a muscle-selective promoter (c) selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof;
one or a plurality of liver-selective enhancer(s) as described above; and
a CK6 promoter or a functional variant thereof.

In a particular embodiment, the nucleic acid molecule of the invention comprises, in particular in this order from 5′ to 3′:
a CK6 promoter or a functional variant thereof;
one or a plurality of liver-selective enhancer(s) as described above; and
a muscle-selective promoter (c) selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof.

According to a particular variant of this embodiment, the nucleic acid molecule of the invention consists of the sequence shown in SEQ ID NO:32, or a functional variant thereof having a sequence at least 80% identical to SEQ ID NO:32, such as at least 85%, 90%, 91%, 92%, 93%, 94%, 95%, 96%, 97%, 98% or even at least 99% identical to SEQ ID NO:32.

In a particular embodiment, the nucleic acid molecule of the invention comprises, in particular in this order from 5′ to 3′:
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter or a functional variant thereof;
one or a plurality of liver-selective enhancer(s) as described above; and
a CK6 promoter or a functional variant thereof.

In a particular embodiment, the nucleic acid molecule of the invention comprises, in particular in this order from 5′ to 3′:
one liver-selective enhancer, in particular the HS-CRM8 enhancer as shown in SEQ ID NO:1, or a functional variant thereof;
a CK6 promoter, in particular the CK6 promoter as shown in SEQ ID NO: 7 or a functional variant thereof; and
a muscle-selective promoter selected in the group consisting of: a spC5-12 CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof.

In a particular embodiment, the nucleic acid molecule of the invention comprises, in particular in this order from 5′ to 3′:
one liver-selective enhancer, in particular the HS-CRM8 enhancer as shown in SEQ ID NO:1, or a functional variant thereof;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof; and
a CK6 promoter, in particular the CK6 promoter as shown in SEQ ID NO:7 or a functional variant thereof.

In a particular embodiment, the nucleic acid molecule of the invention comprises, in particular in this order from 5′ to 3′:
one liver-selective enhancer, in particular the HS-CRM8 enhancer as shown in SEQ ID NO:1, or a functional variant thereof;
a muscle-selective promoter selected in the group consisting of: CK6 promoter, CK8 promoter, MCK promoter, Acta1 promoter, desmin promoter, and functional variants thereof; and
a CK6 promoter, in particular the CK6 promoter as shown in SEQ ID NO:7 or a functional variant thereof.

In a particular embodiment, the nucleic acid molecule of the invention comprises, in particular in this order from 5′ to 3′:
two liver-selective enhancers, in particular two repeats of the HS-CRM8 enhancer as shown in SEQ ID NO:1, or a functional variant thereof;
a CK6 promoter, in particular the CK6 promoter as shown in SEQ ID NO:7 or a functional variant; and
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof.

In a particular embodiment, the nucleic acid molecule of the invention comprises, in particular in this order from 5′ to 3′:
two liver-selective enhancers, in particular two repeats of the HS-CRM8 enhancer as shown in SEQ ID NO:1, or a functional variant thereof;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof; and
a CK6 promoter, in particular the CK6 promoter as shown in SEQ ID NO:7 or a functional variant thereof.

In a particular embodiment, the nucleic acid molecule of the invention comprises, in particular in this order from 5′ to 3′:
two liver-selective enhancers, in particular two repeats of the HS-CRM8 enhancer as shown in SEQ ID NO:1, or a functional variant thereof;
a muscle-selective promoter selected in the group consisting of: CK6 promoter, CK8 promoter, MCK promoter, Acta1 promoter, desmin promoter, and functional variants thereof; and
a CK6 promoter, in particular the CK6 promoter as shown in SEQ ID NO:7 or a functional variant thereof.

In a particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a CK6 promoter, in particular the CK6 promoter as shown in SEQ ID NO:7 or a functional variant thereof; and
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof.

In a particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a CK6 promoter, in particular the CK6 promoter as shown in SEQ ID NO:7 or a functional variant thereof; and
a spC5-12 promoter, in particular the spC5-12 promoter as shown in SEQ ID NO: 4, 5 or 6, in particular as shown in SEQ ID NO:6 or a functional variant thereof.

According to a particular variant of this embodiment, the nucleic acid molecule of the invention consists of the sequence shown in SEQ ID NO:29, or a functional variant thereof having a sequence at least 80% identical to SEQ ID NO:29, such as at least 85%, 90%, 91%, 92%, 93%, 94%, 95%, 96%, 97%, 98% or even at least 99% identical to SEQ ID NO:29.

According to another particular variant, the nucleic acid molecule of the invention consists of the sequence shown in SEQ ID NO:30, or a functional variant thereof having a sequence at least 80% identical to SEQ ID NO:30, such as at least 85%, 90%, 91%, 92%, 93%, 94%, 95%, 96%, 97%, 98% or even at least 99% identical to SEQ ID NO:30.

In a particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a muscle-selective promoter selected in the group consisting of: a spC5-12 promoter, CK6 promoter, CK8 promoter, MCK promoter, Acta1 promoter, desmin promoter, and functional variants thereof, preferably a spC5-12 promoter, in particular the spC5-12 promoter as shown in SEQ ID NO: 4, 5 or 6, in particular as shown in SEQ ID NO:6 or a functional variant thereof; and
a CK6 promoter, in particular the CK6 promoter as shown in SEQ ID NO:7 or a functional variant thereof.

In a particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a muscle-selective promoter selected in the group consisting of: CK6 promoter, CK8 promoter, MCK promoter, Acta1 promoter, desmin promoter, and functional variants thereof; and
a CK6 promoter, in particular the CK6 promoter as shown in SEQ ID NO:7 or a functional variant thereof.

In another particular embodiment, the nucleic acid molecule of the invention comprises, in particular in this order from 5′ to 3′:
one or a plurality of liver-selective enhancer(s) as described above;
a CK6 promoter, in particular the CK6 promoter as shown in SEQ ID NO:7 or a functional variant thereof; and
a spC5-12 promoter, in particular the spC5-12 promoter as shown in SEQ ID NO: 4, 5 or 6, in particular as shown in SEQ ID NO:6 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is selected in the group consisting of: ApoE enhancer, enhancer A3 (SEQ ID NO:40), enhancer F (SEQ ID NO:41), enhancer S1 (SEQ ID NO: 42), enhancer S2 (SEQ ID NO:43), HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14, preferably the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), and HS-CRM8;
a CK6 promoter, in particular the CK6 promoter as shown in SEQ ID NO:7 or a functional variant thereof; and
a spC5-12 promoter, in particular the spC5-12 promoter as shown in SEQ ID NO: 4, 5 or 6, in particular as shown in SEQ ID NO:6 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is HS-CRM8,
a CK6 promoter, in particular the CK6 promoter as shown in SEQ ID NO:7 or a functional variant thereof; and
a spC5-12 promoter, in particular the spC5-12 promoter as shown in SEQ ID NO: 4, 5 or 6, in particular as shown in SEQ ID NO:6 or a functional variant thereof.

In another particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a CK6 promoter, in particular the CK6 promoter as shown in SEQ ID NO:7 or a functional variant thereof; and
a spC5-12 promoter, in particular the spC5-12 promoter as shown in SEQ ID NO: 4, 5 or 6, in particular as shown in SEQ ID NO:6 or a functional variant thereof.

According to a particular variant, the nucleic acid molecule of the invention consists of the sequence shown in SEQ ID NO:30 or a functional variant thereof having a sequence at least 80% identical to SEQ ID NO:30, such as at least 85%, 90%, 91%, 92%, 93%, 94%, 95%, 96%, 97%, 98% or even at least 99% identical to SEQ ID NO:30. The sequence of SEQ ID NO:30 comprises, operably linked to each other: three repeats of the HS-CRM8 enhancer, a CK6 promoter of SEQ ID NO:7 and a spC5-12 promoter of SEQ ID NO:6.

In another particular embodiment, the nucleic acid molecule of the invention comprises, in particular in this order from 5′ to 3′:
one or a plurality of liver-selective enhancer(s) as described above;
a CK6 promoter, in particular the CK6 promoter as shown in SEQ ID NO:7 or a functional variant thereof; and
a CK8 promoter, in particular the CK8 promoter as shown in SEQ ID NO:33 or SEQ ID NO: 34 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is selected in the group consisting of: ApoE enhancer, enhancer A3 (SEQ ID NO:40), enhancer F (SEQ ID NO:41), enhancer S1 (SEQ ID NO: 42), enhancer S2 (SEQ ID NO:43), HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14, preferably the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), and HS-CRM8;
a CK6 promoter, in particular the CK6 promoter as shown in SEQ ID NO:7 or a functional variant thereof; and
a CK8 promoter, in particular the CK8 promoter as shown in SEQ ID NO:33 or SEQ ID NO: 34 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is HS-CRM8,
a CK6 promoter, in particular the CK6 promoter as shown in SEQ ID NO:7 or a functional variant thereof; and
a CK8 promoter, in particular the CK8 promoter as shown in SEQ ID NO:33 or SEQ ID NO: 34 or a functional variant thereof.

In another particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a CK6 promoter, in particular the CK6 promoter as shown in SEQ ID NO:7 or a functional variant thereof; and
a CK8 promoter, in particular the CK8 promoter as shown in SEQ ID NO:33 or SEQ ID NO: 34 or a functional variant thereof.

According to a particular variant, the nucleic acid molecule of the invention consists of the sequence shown in SEQ ID NO:44 or a functional variant thereof having a sequence at least 80% identical to SEQ ID NO:44, such as at least 85%, 90%, 91%, 92%, 93%, 94%, 95%, 96%, 97%, 98% or even at least 99% identical to SEQ ID NO:44. The sequence of SEQ ID NO:44 comprises, operably linked to each other: three repeats of the HS-CRM8 enhancer, a CK6 promoter of SEQ ID NO:7 and a CK8 promoter of SEQ ID NO:33.

In another particular embodiment, the nucleic acid molecule of the invention comprises, in particular in this order from 5′ to 3′:
one or a plurality of liver-selective enhancer(s) as described above;
a first CK6 promoter, in particular the CK6 promoter as shown in SEQ ID NO:7 or a functional variant thereof; and
a second CK6 promoter, in particular the CK6 promoter as shown in SEQ ID NO:7 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is selected in the group consisting of: ApoE enhancer, enhancer A3 (SEQ ID NO:40), enhancer F (SEQ ID NO:41), enhancer S1 (SEQ ID NO: 42), enhancer S2 (SEQ ID NO:43), HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14, preferably the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), and HS-CRM8;
a first CK6 promoter, in particular the CK6 promoter as shown in SEQ ID NO:7 or a functional variant thereof; and
a second CK6 promoter, in particular the CK6 promoter as shown in SEQ ID NO:7 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is HS-CRM8,
a first CK6 promoter, in particular the CK6 promoter as shown in SEQ ID NO:7 or a functional variant thereof; and
a second CK6 promoter, in particular the CK6 promoter as shown in SEQ ID NO:7 or a functional variant thereof.

In another particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a first CK6 promoter, in particular the CK6 promoter as shown in SEQ ID NO:7 or a functional variant thereof; and
a second CK6 promoter, in particular the CK6 promoter as shown in SEQ ID NO:7 or a functional variant thereof.

According to a particular variant, the nucleic acid molecule of the invention consists of the sequence shown in SEQ ID NO:45 or a functional variant thereof having a sequence at least 80% identical to SEQ ID NO:45, such as at least 85%, 90%, 91%, 92%, 93%, 94%, 95%, 96%, 97%, 98% or even at least 99% identical to SEQ ID NO:45. The sequence of SEQ ID NO:45 comprises, operably linked to each other: three repeats of the HS-CRM8 enhancer, a first CK6 promoter of SEQ ID NO:7 and a second CK6 promoter of SEQ ID NO:7.

In another particular embodiment, the nucleic acid molecule of the invention comprises, in particular in this order from 5′ to 3′:
one or a plurality of liver-selective enhancer(s) as described above;
a CK6 promoter, in particular the CK6 promoter as shown in SEQ ID NO:7 or a functional variant thereof; and
a Acta1 promoter, in particular the Acta1 promoter as shown in SEQ ID NO:37 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is selected in the group consisting of: ApoE enhancer, enhancer A3 (SEQ ID NO:40), enhancer F (SEQ ID NO:41), enhancer S1 (SEQ ID NO: 42), enhancer S2 (SEQ ID NO:43), HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14, preferably the liver-selective enhancer is selected in the group consisting of ApoE enhancer, enhancer A3 (SEQ ID NO:40), enhancer F (SEQ ID NO:41), enhancer S1 (SEQ ID NO: 42), enhancer S2 (SEQ ID NO:43), and HS-CRM8;
a CK6 promoter, in particular the CK6 promoter as shown in SEQ ID NO:7 or a functional variant thereof; and
a Acta1 promoter, in particular the Acta1 promoter as shown in SEQ ID NO:37 or a functional variant thereof.

In another particular embodiment, the nucleic acid molecule of the invention comprises, in particular in this order from 5′ to 3′:
one or a plurality of liver-selective enhancer(s), wherein the liver-selective enhancer(s) is HS-CRM8,
a CK6 promoter, in particular the CK6 promoter as shown in SEQ ID NO:7 or a functional variant thereof; and
a Acta1 promoter, in particular the Acta1 promoter as shown in SEQ ID NO:37 or a functional variant thereof.

In another particular embodiment, the nucleic acid molecule of the invention comprises, in particular in this order from 5′ to 3′:
three liver-selective enhancers, in particular three repeats of the HS-CRM8 enhancer as shown in SEQ ID NO:1, or a functional variant thereof;
a CK6 promoter, in particular the CK6 promoter as shown in SEQ ID NO:7 or a functional variant thereof; and
a Acta1 promoter, in particular the Acta1 promoter as shown in SEQ ID NO:37 or a functional variant thereof.

According to a particular variant, the nucleic acid molecule of the invention consists of the sequence shown in SEQ ID NO:46 or a functional variant thereof having a sequence at least 80% identical to SEQ ID NO:46, such as at least 85%, 90%, 91%, 92%, 93%, 94%, 95%, 96%, 97%, 98% or even at least 99% identical to SEQ ID NO:46. The sequence of SEQ ID NO:46 comprises, operably linked to each other: three repeats of the HS-CRM8 enhancer, a CK6 promoter of SEQ ID NO:7 and a Acta1 promoter of SEQ ID NO:37.

In all the embodiments of the nucleic acid molecule of the invention specifically disclosed herein, said nucleic acid molecule may include a linker located between two transcription regulatory elements.

Furthermore, in all the embodiments of the nucleic acid molecule of the invention specifically disclosed herein, said nucleic acid molecule may include a linker located between two liver-selective enhancers within a plurality of liver-selective enhancers. For example in an embodiment comprising a plurality of liver-selective enhancers made of two liver-selective enhancers, a linker may be located or not between these two liver-selective enhancers. In addition, in an embodiment wherein the plurality of liver-selective enhancers comprises three liver-selective enhancers, a linker may be comprised between the first and second liver-selective enhancers and/or between the second and third liver-selective enhancers. For example, in an embodiment wherein the plurality of liver-selective enhancers comprises three liver-selective enhancers, a linker is located between the first and second liver-selective enhancers, and no linker is located between the second and third liver-selective enhancers. In another variant, in an embodiment with three liver-selective enhancers, no linker is located between the first and second liver-selective enhancers, and a linker is located between the second and third liver-selective enhancers.

### Expression Cassette

The nucleic acid molecule of the invention may be introduced into an expression cassette, designed for providing the expression of a transgene of interest into a tissue of interest.

The expression cassette of the invention thus includes the nucleic acid molecule described above, and a transgene of interest.

The expression cassette may comprise at least one further regulatory sequence capable of further controlling the expression of the therapeutic transgene of interest by decreasing or suppressing its expression in certain tissues that are not of interest, of by stabilizing the mRNA coding for the protein of interest, such as a therapeutic protein, encoded by the transgene of interest. These sequences include, for example, silencers (such as tissue-specific silencers), microRNA target sequences, introns and polyadenylation signals.

In a particular embodiment, the expression cassette of the invention comprises, in this order from 5′ to 3′:
the nucleic acid molecule of the invention;
the transgene of interest; and
a polyadenylation signal.

In a particular variant of this embodiment, an intron may be introduced between the nucleic acid molecule of the invention and the transgene of interest. Alternatively, the intron may be located within the transgene of interest. In a particular embodiment, the intron may be a SV40 intron, such as a SV40 intron consisting of SEQ ID NO:9. In a particular embodiment, the nucleic acid construct comprises a human beta globin b2 (or HBB2) intron such as a HBB2 intron of SEQ ID NO: 10 or SEQ ID NO:11; a coagulation factor IX (FIX) intron such as a FIX intron of SEQ ID NO:12 or SEQ ID NO:13; or a chicken beta-globin intron such as a chicken beta globin intron of SEQ ID NO:14 or SEQ ID NO: 15.

Of course, from the teaching disclosed herein and the general knowledge in the fields of molecular biology and gene therapy, one skilled in the art will be able to select and adapt the enhancer number, enhancer size, promoter size, linker size, and any other element such as further enhancer(s) and an intron according to the size of the transgene of interest incorporated into the expression cassette.

The transgene of interest may be any transgene as described in the “definitions” section above. In addition, specific illustrative transgenes of interest are provided in the following tables, where the transgenes are regrouped by families of neuromuscular disorders that they may treat:

Muscular dystrophies

| Gene | Protein |
|---|---|
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge |
| | domain containing 1 |
| PTRF | Polymerase I and transcript release factor |
| MYOT | Myotilin |
| CAV3 | Caveolin 3 |
| DNAJB6 | HSP-40 homologue, subfamily B, number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

Congenital muscular dystrophies

| Gene | Protein |
|---|---|
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT1 | O-linked mannose beta1,2-N-acetylglucosaminyltransferase |
| ISPD | Isoprenoid synthase domain containing |
| POMGNT2 | protein O-linked mannose N-acetylglucosaminyltransferase |
| | 2 |
| B3GNT1 | UDP-GlcNAc:betaGal beta-1,3-N-acetylglucosaminyl- |
| | transferase 1 |
| GMPPB | GDP-mannose pyrophosphorylase B |
| LARGE | Like-glycosyltransferase |
| DPM1 | Dolichyl-phosphate mannosyltransferase 1, catalytic subunit |
| DPM2 | Dolichyl-phosphate mannosyltransferase polypeptide 2, |
| | regulatory subunit |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

Congenital myopathies

| Gene | Protein |
|---|---|
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like (3-Hydroxyacyl-CoA |
| | dehydratase |
| CACNA1S | Calcium channel, voltage-dependent, L type, alpha 1S |
| | subunit |

Distal myopathies

| Gene | |
|---|---|
| symbol | protein |
| DYSF | Dysferlin |
| TTN | Titin |
| GNE | UDP-N-acetylglucosamine-2- epimerase/N-acetylmannosamine |
| | kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein - 280) |
| VCP | Valosin-containing protein |

Other myopathies

| Gene | |
|---|---|
| symbol | protein |
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein - 280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

Myotonic syndromes

| Gene | protein |
|---|---|
| DMPK | Myotonic dystrophy protein kinase |
| CNPB | Cellular nucleic acid-binding protein |
| CLCN1 | Chloride channel 1, skeletal muscle (Thomsen disease, |
| | autosomal dominant) |
| CAV3 | Caveolin 3 |
| HSPG2 | Perlecan |
| ATP2A1 | ATPase, Ca++ transporting, fast twitch 1 |

Ion Channel muscle diseases

| Gene | protein |
|---|---|
| CLCN1 | Chloride channel 1, skeletal muscle (Thomsen disease, |
| | autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, alpha 1S |
| | subunit |
| CACNA1A | Calcium channel, voltage-dependent, P/Q type, alpha 1A |
| | subunit |
| KCNE3 | Potassium voltage-gated channel, Isk-related family, |
| | member 3 |
| KCNA1 | Potassium voltage-gated channel, shaker-related subfamily, |
| | member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, member 2 |
| KCNQ1 | Potassium voltage-gated channel, KQT-like subfamily, |
| | member 1 |
| KCNE2 | Potassium voltage-gated channel, Isk-related family, |
| | member 2 |
| KCNE1 | Potassium voltage-gated channel, Isk-related family, |
| | member 1 |

Malignant hyperthermia

| Gene | protein |
|---|---|
| RYR1 | Ryanodine receptor 1 (skeletal) |
| CACNA1S | Calcium channel, voltage-dependent, L type, alpha |
| | 1S subunit |

Metabolic myopathies

| Gene | protein |
|---|---|
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen |
| | branching enzyme, Andersen disease, glycogen storage |
| | disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) |
| | glycogen synthase 1 (muscle) |
| PRKAG2 | Protein kinase, AMP-activated, gamma 2 non-catalytic |
| | subunit |
| RBCK1 | RanBP-type and C3HC4-type zinc finger containing 1 |
| | (heme-oxidized IRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

Hereditary Cardiomyopathies

| Gene | protein |
|---|---|
| MYH6 | Myosin heavy chain 6 |
| MYH7 | Myosin, heavy polypeptide 7, cardiac muscle, beta |
| TNNT2 | Troponin T2, cardiac |
| TPM1 | Tropomyosin 1 (alpha) |
| MYBPC3 | Cardiac myosin binding protein-C |
| PRKAG2 | Protein kinase, AMP-activated, gamma 2 non-catalytic |
| | subunit |
| TNNI3 | Troponin I, cardiac |
| MYL3 | Myosin light chain 3 |
| TTN | Titin |
| MYL2 | Myosin light chain 2 |
| ACTC1 | Actin, alpha, cardiac muscle precursor |
| CSRP3 | Cysteine and glycine-rich protein 3 (cardiac LIM protein) |
| TNNC1 | Slow troponin C |
| VCL | Vinculin |
| MYLK2 | Myosin light chain kinase 2 |
| CAV3 | Caveolin 3 |
| MYOZ2 | Myozenin 2, or calsarcin 1, a Z disk protein |
| JPH2 | Junctophilin-2 |
| PLN | Phospholamban |
| NEXN | Nexilin(F-actin binding protein) |
| ANKRD1 | Ankyrin repeat domain 1 (cardiac muscle) |
| ACTN2 | Actinin alpha2 |
| NDUFAF1 | NADH-ubiquinone oxidoreductase 1 alpha subcomplex |
| TSFM | Ts translation elongation factor, mitochondrial |
| AARS2 | Alanyl-tRNA synthetase 2, mitochondrial |
| MRPL3 | Mitochondrial ribosomal protein L3 |
| COX15 | COX15 homolog, cytochrome c oxidase assembly protein |
| | (yeast) |
| MTO1 | Mitochondrial tRNA translation optimization 1 |
| MRPL44 | Mitochondrial ribosomal protein L44 |
| LMNA | Lamin A/C |
| LDB3 | LIM domain binding 3 |
| SCN5A | Voltage-gated sodium channel type V alpha |
| DES | Desmin |
| EYA4 | Eyes absent 4 |
| SGCD | Delta-sarcoglycan |
| TCAP | Telethonin |
| ABCC9 | ATP-binding cassette, sub-family C (member 9) |
| TMPO | Lamina-associated polypeptide 2 |
| PSEN2 | Presenilin 2 |
| CRYAB | Crystallin, alpha B |
| FKTN | Fukutin |
| TAZ | Tafazzin |
| DMD | Dystrophin |
| LAMA4 | Laminin alpha 4 |
| ILK | Integrin-linked kinase |
| MYPN | Myopalladin |
| RBM20 | RNA binding motif protein 20 |
| SYNE1 | Spectrin repeat containing, nuclear envelope 1 (nesprin 1) |
| MURC | Muscle-related coiled-coil protein |
| DOLK | Dolichol kinase |
| GATAD1 | GATA zinc finger domain containing 1 |
| SDHA | succinate dehydrogenase complex, subunit A, |
| | flavoprotein (Fp) |
| GAA | Acid alpha-glucosidase preproprotein |
| DTNA | Dystrobrevin, alpha |
| FLNA | Filamin A, alpha (actin binding protein 280) |
| TGFB3 | Transforming growth factor, beta 3 |
| RYR2 | Ryanodine receptor 2 |
| TMEM43 | Transmembrane protein 43 |
| DSP | Desmoplakin |
| PKP2 | Plakophilin 2 |
| DSG2 | Desmoglein 2 |
| DSC2 | Desmocollin 2 |
| JUP | Junction plakoglobin |
| CASQ2 | Calsequestrin 2 (cardiac muscle) |
| KCNQ1 | Potassium voltage-gated channel, KQT-like subfamily, |
| | member 1 |
| KCNH2 | Voltage-gated potassium channel, subfamily H, member 2 |
| ANK2 | Ankyrin 2 |
| KCNE1 | Potassium voltage-gated channel, Isk-related family, |
| | member 1 |
| KCNE2 | Potassium voltage-gated channel, Isk-related family, |
| | member 2 |
| KCNJ2 | Potassium inwardly-rectifying channel J2 |
| CACNA1C | Calcium channel, voltage-dependent, L type, alpha 1C |
| | subunit |
| SCN4B | Sodium channel, voltage-gated, type IV, beta subunit |
| AKAP9 | A kinase (PRKA) anchor protein (yotiao) 9 |
| SNTA1 | Syntrophin, alpha 1 |
| KCNJ5 | Potassium inwardly-rectifying channel, subfamily J, |
| | member 5 |
| NPPA | Natriuretic peptide precursor A |
| KCNA5 | Potassium voltage-gated channel, shaker-related subfamily, |
| | member 5 |
| GJA5 | Connexin 40 |
| SCN1B | Sodium channel, voltage-gated, type I, beta subunit |
| SCN2B | Sodium channel, voltage-gated, type II, beta subunit |
| NUP155 | Nucleoporin 155 kDa |
| GPD1L | Glycerol-3-phosphate dehydrogenase 1-like |
| CACNB2 | Calcium channel, voltage-dependent, beta 2 subunit |
| KCNE3 | Potassium voltage-gated channel, Isk-related family, |
| | member 3 |
| SCN3B | Sodium channel, voltage-gated, type III, beta subunit |
| HCN4 | Hyperpolarization activated cyclic nucleotide-gated |
| | potassium channel 4 |

Congenital myasthenic syndromes

| Gene | protein |
|---|---|
| CHRNA1 | Cholinergic receptor, nicotinic, alpha polypeptide 1 |
| CHRNB1 | Cholinergic receptor, nicotinic, beta 1 muscle |
| CHRND | Cholinergic receptor, nicotinic, delta |
| CHRNE | Cholinergic receptor, nicotinic, epsilon |
| RAPSN | Rapsyn |
| CHAT | Choline acetyltransferase isoform |
| COLQ | Acetylcholinesterase collagen-like tail subunit |
| MUSK | muscle, skeletal, receptor tyrosine kinase |
| DOK7 | Docking protein 7 |
| AGRN | Agrin |
| GFPT1 | Glutamine-fructose-6-phosphate transaminase 1 |
| DPAGT1 | Dolichyl-phosphate (UDP-N-acetylglucosamine) N- |
| | acetylglucosaminephosphotransferase 1 (GlcNAc-1-P |
| | transferase) |
| LAMB2 | Laminin, beta 2 (laminin S) |
| SCN4A | Sodium channel, voltage-gated, type IV, alpha |
| CHRNG | Cholinergic receptor, nicotinic, gamma polypeptide |
| PLEC | plectin |
| ALG2 | Alpha-1,3/1,6-mannosyltransferase |
| ALG14 | UDP-N-acetylglucosaminyltransferase |
| SYT2 | Synaptotagmin II |
| PREPL | Prolyl endopeptidase-like |

Motor Neuron diseases

| Gene | protein |
|---|---|
| SMN1 | Survival of motor neuron 1, telomeric |
| IGHMBP2 | Immunoglobulin mu binding protein 2 |
| PLEKHG5 | Pleckstrin homology domain containing, family G (with |
| | RhoGef domain) member 5 |
| HSPB8 | Heat shock 27 kDa protein 8 |
| HSPB1 | Heat shock 27 kDa protein 1 |
| HSPB3 | Heat shock 27 kDa protein 3 |
| AARS | Alanyl-tRNA synthetase |
| GARS | Glycyl-tRNA synthetase |
| BSCL2 | Seipin |
| REEP1 | Receptor accessory protein 1 |
| SLC5A7 | Solute carrier family 5 (sodium/choline cotransporter), |
| | member 7 |
| DCTN1 | Dynactin 1 |
| UBA1 | Ubiquitin-activating enzyme 1 |
| ATP7A | ATPase, Cu++ transporting, alpha polypeptide |
| DNAJB2 | DnaJ (Hsp40) homolog, subfamily B, member 2 |
| TRPV4 | Transient receptor potential cation channel, subfamily V, |
| | member 4 |
| DYNC1H1 | Dynein, cytoplasmic 1, heavy chain 1 |
| BICD2 | Bicaudal D homolog 2 (Drosophila) |
| FBXO38 | F-box protein 38 |
| ASAH1 | N-acylsphingosine amidohydrolase (acid ceramidase) 1 |
| VAPB | Vesicle-associated membrane protein-associated protein B |
| | and C |
| EXOSC8 | Exosome component 8 |
| SOD1 | Superoxide dismutase 1, soluble |
| ALS2 | Alsin |
| SETX | Senataxin |
| FUS | Fusion (involved in t(12; 16) in malignant liposarcoma) |
| ANG | Angiogenin |
| TARDBP | TAR DNA binding protein |
| FIG4 | Sac domain-containing inositol phosphatase 3 |
| OPTN | Optineurin |
| ATXN2 | Ataxin 2 |
| VCP | Valosin-containing protein |
| UBQLN2 | Ubiquilin 2 |
| SIGMAR1 | Sigma non-opioid intracellular receptor 1 |
| CHMP2B | Charged multivesicular body protein 2B |
| PFN1 | Profilin 1 |
| MATR3 | Matrin 3 |
| NEFH | Neurofilament, heavy polypeptide |
| PRPH | Peripherin |
| C9orf72 | Chromosome 9 open reading frame 72 |
| CHCHD10 | Coiled-coil-helix-coiled-coil-helix domain containing 10 |
| SQSTM1 | Sequestosome 1 |
| AR | Androgen receptor |
| GLE1 | GLE1 RNA export mediator homolog (yeast) |
| ERBB3 | V-erb-b2 erythroblastic leukemia viral oncogene homolog 3 |
| | (avian) |
| PIP5K1C | Phosphatidylinositol-4-phosphate 5-kinase, type I, gamma |
| EXOSC3 | Exosome component 3 |
| VRK1 | Vaccinia related kinase 1 |
| SLC52A3 | Solute carrier family 52, riboflavin transporter, member 3 |
| SLC52A2 | Solute carrier family 52, riboflavin transporter, member 2 |
| HEXB | Hexosaminidase B |

Hereditary motor and sensory neuropathies

| Gene | Protein |
|---|---|
| PMP22 | Peripheral myelin protein 22 |
| MPZ | Myelin protein zero |
| LITAF | Lipopolysaccharide-induced TNF factor |
| EGR2 | Early growth response 2 protein |
| NEFL | Neurofilament, light polypeptide 68 kDa |
| HOXD10 | Homeobox D10 |
| ARHGEF10 | Rho guanine nucleotide exchange factor 10 |
| FBLN5 | Fibulin 5 (extra-cellular matrix) |
| DNM2 | Dynamin 2 |
| YARS | Tyrosyl-tRNA synthetase |
| INF2 | Inverted formin 2 |
| GNB4 | Guanine nucleotidebinding protein, beta-4 |
| GDAP1 | Ganglioside-induced differentiation-associated protein 1 |
| MTMR2 | Myotubularin-related protein 2 |
| SBF2 | SET binding factor 2 |
| SBF1 | SET binding factor 1 |
| SH3TC2 | KIAA1985 protein |
| NDRG1 | N-myc downstream regulated gene 1 |
| PRX | Periaxin |
| HK1 | Hexokinase 1 |
| FGD4 | Actin-filament binding protein Frabin |
| FIG4 | Sac domain-containing inositol phosphatase 3 |
| SURF1 | surfeit 1 |
| GJB1 | Gap junction protein, beta 1, 32 kDa (connexin 32) |
| AIFM1 | Apoptosis-inducing factor, mitochondrionassociated 1 |
| PRPS1 | Phosphoribosyl pyrophosphate synthetase 1 |
| PDK3 | Pyruvate dehydrogenase kinase, isoenzyme 3 |
| KIF1B | Kinesin family member 1B |
| MFN2 | Mitofusin 2 |
| RAB7A | RAB7, member RAS oncogene family |
| TRPV4 | Transient receptor potential cation channel, subfamily V, |
| | member 4 |
| GARS | Glycyl-tRNA synthetase |
| HSPB1 | Heat shock 27 kDa protein 1 |
| HSPB8 | Heat shock 27 kDa protein 8 |
| AARS | Alanyl-tRNA synthetase |
| DYNC1H1 | Dynein, cytoplasmic 1, heavy chain 1 |
| LRSAM1 | leucine rich repeat and sterile alpha motif containing 1 |
| DHTKD1 | dehydrogenase E1 and transketolase domain containing 1 |
| TRIM2 | Tripartite motif containing 2 |
| TFG | TRK-fused gene |
| MARS | methionyl-tRNA synthetase |
| KIF5A | Kinesin family member 5A |
| LMNA | Lamin A/C |
| MED25 | Mediator complex subunit 25 |
| DNAJB2 | DnaJ (Hsp40) homolog, subfamily B, member 2 |
| HINT1 | Histidine triad nucleotide binding protein 1 |
| KARS | Lysyl-tRNA synthetase |
| PLEKHG5 | Pleckstrin homology domain containing, family G (with |
| | RhoGef domain) member 5 |
| COX6A1 | Cytochrome c oxidase subunit VIa polypeptide 1 |
| IGHMBP2 | Immunoglobulin mu binding protein 2 |
| SPTLC1 | Serine palmitoyltransferase subunit 1 |
| SPTLC2 | Serine palmitoyltransferase long chain base subunit 2 |
| ATL1 | Atlastin GTPase 1 |
| KIF1A | Kinesin family member 1A |
| WNK1 | WNK lysine deficient protein kinase 1 |
| IKBKAP | Inhibitor of kappa light polypeptide gene enhancer in |
| | B-cells, kinase complex-associated protein |
| NGF | Nerve growth factor (beta polypeptide) |
| DNMT1 | DNA (cytosine-5)-methyltransferase 1 |
| SLC12A6 | Potassium chloride cotransporter KCC3 |
| GJB3 | Gap junction protein, beta 3, 31 kDa (=connexin 31) |
| sept-09 | Septin 9 |
| GAN | Gigaxonin |
| CTDP1 | CTD phosphatase subunit 1 |
| VRK1 | Vaccinia related kinase 1 |

Hereditary paraplegia

| Gene | |
|---|---|
| symbol | protein |
| ATL1 | Atlastin |
| SPAST | Spastin |
| NIPA1 | Non-imprinted in Prader-Willi/Angelman syndrome 1 |
| KIAA0196 | Strumpellin |
| KIF5A | Kinesin family member 5A |
| RTN2 | Reticulon 2 |
| HSPD1 | Heat shock 60 kDa protein 1 (chaperonin) |
| BSCL2 | Seipin |
| REEP1 | Receptor accessory protein 1 |
| ZFYVE27 | Protrudin |
| SLC33A1 | Solute carrier family 33 (acetyl- CoA transporter) |
| CYP7B1 | Cytochrome P450, family 7, subfamily B, polypeptide 1 |
| SPG7 | Paraplegin |
| SPG11 | Spatacsin |
| ZFYVE26 | Spastizin |
| ERLIN2 | ER lipid raft associated 2 |
| SPG20 | Spartin |
| SPG21 | Maspardin |
| B4GALNT1 | beta-1,4-N-acetyl-galactosaminyl transferase 1 |
| DDHD1 | DDHD domain containing 1 |
| KIF1A | Kinesin family member 1A |
| FA2H | Fatty acid 2-hydroxylase |
| PNPLA6 | Patatin-like phospholipase domain containing 6 |
| C19orf12 | chromosome 19 open reading frame 12 |
| GJC2 | gap junction protein, gamma 2, 47 kDa |
| NT5C2 | 5′-nucleotidase, cytosolic II |
| GBA2 | glucosidase, beta (bile acid) 2 |
| AP4B1 | adaptor-related protein complex 4, beta 1 subunit |
| AP5Z1 | Hypothetical protein LOC9907 |
| TECPR2 | tectonin beta-propeller repeat containing 2 |
| AP4M1 | Adaptor-related protein complex 4, mu 1 subunit |
| AP4E1 | Adaptor-related protein complex 5, zeta 1 subunit |
| AP4S1 | adaptor-related protein complex 4, sigma 1 subunit |
| DDHD2 | DDHD domain containing 2 |
| C12orf65 | adaptor-related protein complex 4, sigma 1 subunit |
| CYP2U1 | cytochrome P450, family 2, subfamily U, polypeptide 1 |
| ARL6IP1 | ADP-ribosylation factor-like 6 interacting protein 1 |
| AMPD2 | adenosine monophosphate deaminase 2 |
| ENTPD1 | ectonucleoside triphosphate diphosphohydrolase 1 |
| ALDH3A2 | Aldehyde dehydrogenase 3A2 |
| ALS2 | Alsin |
| L1CAM | L1 cell adhesion molecule |
| PLP1 | Proteolipid protein 1 |
| MTPAP | mitochondrial poly(A) polymerase |
| AFG3L2 | AFG3 ATPase family gene 3-like 2 (S. cerevisiae) 1 |
| SACS | Sacsin |

Other neuromuscular disorders

| Gene | protein |
|---|---|
| TOR1A | Torsin A |
| SGCE | Sarcoglycan, epsilon |
| IKBKAP | Inhibitor of kappa light polypeptide gene enhancer in B- |
| | cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain containing 10 |
| CASQ1 | Calsequestrin 1 (fast-twitch, skeletal muscle) |
| YARS2 | tyrosyl-tRNA synthetase 2, mitochondrial |

In particular embodiment, the transgene of interest is: α-L-iduronidase, acid-α-glucosidase (GAA), Glycogen Debranching Enzyme (GDE) or shortened forms of GDE, G6P, alpha-sarcoglycan (SGCA), dystrophin or its shortened forms; or SMN1.

In a particular embodiment, the transgene has a length of at most 3500 bp.

### Vectors, Cells and Pharmaceutical Compositions

The expression cassette of the invention may be introduced into a vector. Thus, the invention also relates to a vector comprising the expression cassette described above. The vector used in the present invention is a vector suitable for RNA/protein expression, and in particular suitable for gene therapy.

In one embodiment, the vector is a plasmid vector.

In another embodiment, the vector is a non-viral vector, such as a nanoparticle, a lipid nanoparticle (LNP) or a liposome, containing the expression cassette of the invention.

In another embodiment, the vector is a system based on transposons, allowing integration of the expression cassette of the invention in the genome of the target cell, such as the hyperactive Sleeping Beauty (SB100X) transposon system (Mates et al. 2009).

In a further embodiment, the transgene of interest is a repair matrix useful for targeted genome engineering, such as a repair matrix suitable for the correction of a gene along with an endonuclease as described above. More particularly, the vector includes a repair matrix containing arms of homology to a gene of interest, for homology driven integration.

In another embodiment, the vector is a viral vector suitable for gene therapy, targeting muscles and/or the CNS. In this case, the further sequences are added to the expression cassette of the invention, suitable for producing an efficient viral vector, as is well known in the art. In a particular embodiment, the viral vector is derived from an integrating virus. In particular, the viral vector may be derived from an adenovirus, a retrovirus or a lentivirus (such as an integration-deficient lentivirus). In a particular embodiment, the lentivirus is a pseudotyped lentivirus having an enveloped that enable the targeting of cells/tissues of interest, such as muscle cells (as described in patent applications EP17306448.6 and EP17306447.8). In case the viral vector is derived from a retrovirus or lentivirus, the further sequences are retroviral or lentiviral LTR sequences flanking the expression cassette. In another particular embodiment, the viral vector is a parvovirus vector, such as an AAV vector, such as an AAV vector suitable for transducing a muscles and/or the CNS. In this embodiment, the further sequences are AAV ITR sequences flanking the expression cassette.

In a preferred embodiment, the vector is an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter). Therefore, AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising capsid with Y44+500+730F+T491V changes, disclosed in Ling et al., 2016 Jul. 18, Hum Gene Ther Methods.), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24 (6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as cy 10 and -rh10, -rh74, -rh74-9 as disclosed in EP18305399 (such as the Hybrid Cap rh74-9 serotype described in examples of EP18305399; a rh74-9 serotype being also referred to herein as “-rh74-9”, “AAVrh74-9” or “AAV-rh74-9”), -9-rh74 as disclosed in EP18305399 (such as the Hybrid Cap 9-rh74 serotype described in the examples of EP18305399; a -9-rh74 serotype being also referred to herein as “−9-rh74”, “AAV9-rh74”, “AAV-9-rh74”, or “rh74-AAV9”), -dj, Anc80, LK03, AAV2i8, porcine AAV serotypes such as AAVpo4 and AAVpo6, and tyrosine, lysine and serine capsid mutants of the AAV serotypes, etc. In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

Desirable AAV fragments for assembly into vectors include the cap proteins, including the VP1, VP2, VP3 and hypervariable regions, the rep proteins, including rep 78, rep 68, rep 52, and rep 40, and the sequences encoding these proteins. These fragments may be readily utilized in a variety of vector systems and host cells.

AAV-based recombinant vectors lacking the Rep protein integrate with low efficacy into the host's genome and are mainly present as stable circular episomes that can persist for years in the target cells.

Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In the context of the present invention, the AAV vector comprises an AAV capsid able to transduce the target cells of interest, i.e. muscle cells and CNS cells. By “CNS” is meant all cells and tissue of the brain and spinal cord. Thus, the term includes, but is not limited to, neuronal cells, glial cells, astrocytes, cerebrospinal fluid (CSF), interstitial spaces, bone, cartilage and the like.

According to a particular embodiment, the AAV vector is selected from the group comprising the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24 (6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as -cy 10 and -rh10, -rh39, -rh43, -rh74, -rh74-9, -dj, Anc80, LK03, AAV.PHP, AAV2i8, porcine AAV such as AAVpo4 and AAVpo6, and tyrosine, lysine and serine capsid mutants of AAV serotypes. In a particular embodiment, the AAV vector is of the AAV8, AAV9, AAVrh74, AAVrh74-9, or AAV2i8 serotype (i.e. the AAV vector has a capsid of the AAV8, AAV9, AAVrh74, AAVrh74-9 or AAV2i8 serotype). In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, in particular AAV2 serotype, and whose capsid is derived from another serotype. For example, the genome of the pseudotyped vector may have a capsid derived from the AAV8, AAV9, AAVrh74, AAVrh74-9, or AAV218 serotype, and its genome may be derived from and different serotype. In a particular embodiment, the AAV vector has a capsid of the AAV8, AAV9, AAVrh74 or AAVrh74-9 serotype, in particular of the AAV8 or AAV9 serotype, more particularly of the AAV8 serotype.

In another embodiment, the capsid is a modified capsid. In the context of the present invention, a “modified capsid” may be a chimeric capsid or capsid comprising one or more variant VP capsid proteins derived from one or more wild-type AAV VP capsid proteins.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. For example, a chimeric AAV vector can derive from the combination of an AAV8 capsid sequence with a sequence of an AAV serotype different from the AAV8 serotype, such as any of those specifically mentioned above.

In another embodiment, the modified capsid can be derived also from capsid modifications inserted by error prone PCR and/or peptide insertion (e.g. as described in Bartel et al., 2011). In addition, capsid variants may include single amino acid changes such as tyrosine mutants (e.g. as described in Zhong et al., 2008). In a particular embodiment, the capsid of the AAV vector is a peptide-modified hybrid between AAV serotype 9 (AAV9) and AAV serotype 74 (AAVrh74) capsid proteins, as described in WO2019/193119 or in WO2020/200499 or in WO2022053630, such as an AAV9-rh74 hybrid capsid or AAVrh74-9 hybrid capsid modified with the P1 peptide. In a particular embodiment, the capsid of the AAV is an AAV9-rh74 capsid as described in WO2019/193119, an AAV9-rh74-P1 capsid as described in WO2020/200499, or an AAV9-rh74-HB-P1 capsid as described in WO2022053630.

In a further embodiment, the AAV vector is an AAV vector as described in WO2020/216861 or an AAV vector as described in WO2022/003211. In particular, the AAV vector may have a variant AAV2 capsid as described in WO2020/216861, or a hybrid capsid between AAV8 and AAV2/13 as described in WO2022/003211.

In a further embodiment, the AAV vector comprises a porcine AAV serotype 1 (AAVpo1) capsid wild-type (or modified with the A1 peptide (AAVpo1-A1) as described in WO2021/219762.

In addition, the genome of the AAV vector may either be a single stranded or self-complementary double-stranded genome (McCarty et al., Gene Therapy, 2003). Self-complementary double-stranded AAV vectors are generated by deleting the terminal resolution site from one of the AAV terminal repeats. These modified vectors, whose replicating genome is half the length of the wild type AAV genome have the tendency to package DNA dimers. In a preferred embodiment, the AAV vector implemented in the practice of the present invention has a single stranded genome, and further preferably comprises an AAV8, AAV9, AAVrh74, AAVrh74-9, or AAV218 capsid, in particular an AAV8, AAV9, AAVrh74 or AAVrh74-9 capsid, such as an AAV8 or AAV9 capsid, more particularly an AAV8 capsid. As is known in the art, additional suitable sequences may be introduced in the nucleic acid construct of the invention for obtaining a functional viral vector. Suitable sequences include AAV ITRs.

Of course, in designing the nucleic acid sequence of the invention and the expression cassette of the invention one skilled in the art will take care of respecting the size limit of the vector used for delivering said construct to a cell or organ. In particular, as reminded above, in case of the vector being an AAV vector one skilled in the art knows that a major limitation of AAV vector is its cargo capacity which may vary from one AAV serotype to another but is thought to be limited to around the size of parental viral genome. For example, 5 kb is the maximum size usually thought to be packaged into an AAV8 capsid. (Wu Z. et al., Mol Ther., 2010, 18 (1): 80-86; Lai Y. et al., Mol Ther., 2010, 18 (1): 75-79; Wang Y. et al., Hum Gene Ther Methods, 2012, 23 (4): 225-33). Accordingly, those skilled in the art will take care in practicing the present invention to select the components of the nucleic acid construct of the invention so that the resulting nucleic acid sequence, including sequences coding AAV 5′- and 3′-ITRs to preferably not exceed 110% of the cargo capacity of the AAV vector implemented, in particular to preferably not exceed 5.5 kb.

The invention also relates to an isolated cell, for example muscle cell or CNS cell, which is transformed with a nucleic acid sequence of the invention or with the expression cassette of the invention. The isolated cell of the invention may be delivered to the subject in need thereof via injection in the tissue of interest or in the bloodstream of said subject. In a particular embodiment, the invention involves introducing the nucleic acid molecule or the expression cassette of the invention into an isolated cell of the subject to be treated, and administering back to the subject said cell into which the nucleic acid or expression cassette has been introduced.

The present invention also provides a pharmaceutical composition comprising a nucleic acid molecule, a vector or an isolated cell of the invention. Such compositions comprise a therapeutically effective amount of the nucleic acid sequence, vector or isolated cell of the invention, and a pharmaceutically acceptable carrier. The term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject. In a particular embodiment, the nucleic acid sequence, expression cassette, vector or isolated cell of the invention is formulated in a composition comprising phosphate-buffered saline and supplemented with 0.25% human serum albumin. In another particular embodiment, the vector of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011.

In a preferred embodiment, the composition is formulated in accordance with routine procedures as a pharmaceutical composition adapted for intravenous or intramuscular administration, preferably intravenous administration, to human beings. Typically, compositions for intravenous administration are solutions in sterile isotonic aqueous buffer. Where necessary, the composition may also include a solubilizing agent and a local anesthetic such as lignocaine to, ease pain at the, site of the injection.

In an embodiment, the nucleic acid sequence, expression cassette or vector of the invention can be delivered in a vesicle, in particular a liposome. In yet another embodiment, the nucleic acid sequence, expression cassette or the vector of the invention can be delivered in a controlled release system.

### Methods of Use

Thanks to the present invention, a transgene of interest may be expressed in muscle and CNS cells.

The nucleic acid molecule, expression cassette or vector of the present invention may be used for expressing a gene into a muscle and/or in CNS cell. Accordingly, the invention provides a method for expressing a transgene of interest in a muscle cell or CNS cell, wherein the expression cassette of the invention is introduced in the cell, and the transgene of interest is expressed. The method may be an in vitro, ex vivo or in vivo method for expressing a transgene of interest in a muscle or CNS cell.

In a particular aspect, the invention relates to the nucleic acid molecule, expression cassette or vector of the present invention for use in an ex-vivo method for expressing a transgene of interest in a cell, wherein the expression cassette of the invention is introduced in the cell, and the transgene of interest is expressed.

The nucleic acid molecule, expression cassette or vector of the present invention may also be used for gene therapy. Accordingly, in one aspect, the invention relates to a nucleic acid molecule, expression cassette, vector, isolated cell or pharmaceutical composition as described above, for use as a medicament. In an aspect, the invention thus relates to the nucleic acid molecule, expression cassette or vector disclosed herein for use in therapy, specifically in gene therapy. Likewise, the isolated cell of the invention may be used in therapy, specifically in cell therapy.

In another aspect, the invention relates to a nucleic acid molecule, expression cassette, vector, isolated cell or pharmaceutical composition as described above, for use in a method for the treatment of a neuromuscular disorder.

In a further aspect, the invention relates to the use of a nucleic acid molecule, expression cassette, vector, isolated cell or pharmaceutical composition as described above, for the manufacture of a medicament for use in the treatment of a neuromuscular disorder.

In another aspect, the invention relates to a method for the treatment of a neuromuscular disorder, comprising administering a therapeutically effective amount of the nucleic acid molecule, expression cassette, vector, isolated cell or pharmaceutical composition described herein to a subject in need thereof.

The neuromuscular disorder is in particular an inherited or acquired disorder, such as an inherited or acquired neuromuscular disease. Of course, the therapeutic transgene and the promoter driving expression into a tissue of therapeutic interest will be selected in view of the disorder to be treated.

The term “neuromuscular disorder” encompasses diseases and ailments that impair the functioning of the muscles, either directly, being pathologies of the voluntary muscle, or indirectly, being pathologies of nerves or neuromuscular junctions. Illustrative neuromuscular disorders include, without limitation, muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy), motor neuron diseases (e.g. amyotrophic lateral sclerosis (ALS), spinal muscular atrophy (Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4)), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphogly cerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenital, paramyotonia congenital, central core disease, nemaline myopathy, myotubular myopathy, periodic paralysis). In this embodiment, the nucleic acid sequence of the invention comprises liver-selective, muscle-selective and/or neuron-selective transcription regulatory elements, such as liver-selective and muscle-selective transcription regulatory elements, liver-selective and neuron-selective transcription regulatory elements, and liver-selective, muscle-selective and neuron-selective transcription regulatory elements

In a particular embodiment, the disorder is a glycogen storage disease. The expression “glycogen storage disease” denotes a group of inherited metabolic disorders involving enzymes responsible for the synthesis and degradation of glycogen. In a more particular embodiment, the glycogen storage disease may be GSDI (von Gierke's disease), GSDII (Pompe disease), GSDIII (Cori disease), GSDIV, GSDV, GSDVI, GSDVII, GSDVIII or lethal congenital glycogen storage disease of the heart. More particularly, the glycogen storage disease is selected in the group consisting of GSDI, GSDII and GSDIII, even more particularly in the group consisting of GSDII and GSDIII. In an even more particular embodiment, the glycogen storage disease is GSDII. In particular, the nucleic acid molecules of the invention may be useful in gene therapy to treat GAA-deficient conditions, or other conditions associated by accumulation of glycogen such as GSDI (von Gierke's disease), GSDII (Pompe disease), GSDIII (Cori disease), GSDIV, GSDV, GSDVI, GSDVII, GSDVIII and lethal congenital glycogen storage disease of the heart, more particularly GSDI, GSDII or GSDIII, even more particularly GSDII and GSDIII. In a further particular embodiment, the disorder is Pompe disease and the therapeutic transgene is a gene encoding an acid alpha-glucosidase (GAA) or a variant thereof. Such variants of GAA are in particular disclosed in applications PCT/2017/072942, PCT/EP2017/072945 and PCT/EP2017/072944, which are incorporated herein by reference in their entirety. In this embodiment, the nucleic acid sequence of the invention comprises liver-selective, muscle-selective and/or neuron-selective transcription regulatory elements, such as liver-selective and muscle-selective transcription regulatory elements, liver-selective and neuron-selective transcription regulatory elements, muscle-selective and neuron-selective transcription regulatory elements, and liver-selective, muscle-selective and neuron-selective transcription regulatory elements. In a particular embodiment, the disorder is infantile-onset Pompe disease (IOPD) or late onset Pompe disease (LOPD). Preferably, the disorder is IOPD.

One skilled in the art is aware of the transgene of interest useful in the treatment of these and other disorders by gene therapy. For example, the therapeutic transgene is: lysosomal enzymes α-L-iduronidase [IDUA (alphase-Liduronidase)], for MPSI, acid-α-glucosidase (GAA) for Pompe disease, Glycogen Debranching Enzyme (GDE) or shortened forms of GDE (also referred to as truncated forms of GDE, or mini-GDE) for Cori disease (GSDIII), G6P for GSDI, alpha-sarcoglycan (SGCA) for LGMD2D; dystrophin or its shortened forms for DMD; and SMN1 for SMA. The transgene of interest may also be a transgene that provides other therapeutic properties than providing a missing protein or a RNA suppressing the expression of a given protein. For example, transgenes of interest may include, without limitation, transgenes that may increase muscle strength.

Specific examples of therapeutic transgenes of interest that may be operably linked to the hybrid promoter of the invention for specific diseases are provided below.

In a particular embodiment, the disease is Cori disease and the transgene of interest encodes a GDE or a shortened form of GDE. Shortened forms of GDE suitable for use in the present invention may include, without limitation, those described in EP18306088. Alternatively, the present invention is used in a dual AAV vector system for expressing GDE, such as the dual AAV vector system disclosed in WO2018162748. In this embodiment, the vector of the present invention may correspond to the first AAV vector of the dual AAV vector system, comprising between 5′ and 3′ AAV ITRs, a first nucleic acid sequence that encodes a N-terminal part of a GDE under the control of a nucleic acid molecule of the present invention.

In another particular embodiment, the disease is Pompe disease, and the transgene of interest encodes an acid-α-glucosidase (GAA), or a modified GAA. Modified GAA suitable for use in the present invention include, without limitation, those disclosed in WO2018046772, WO2018046774 and WO2018046775.

In a further particular embodiment, the disorder is selected from Duchene muscular dystrophy, myotubular myopathy, spinal muscular atrophy, limb-girdle muscular dystrophy type 2I, 2A, 2B, 2C or 2D and myotonic dystrophy type 1.

Methods of administration of the vector of the invention include but are not limited to intradermal, intramuscular, intraperitoneal, intravenous, subcutaneous, intranasal, epidural, locoregional administration as described in WO2015158924 and oral routes. In a particular embodiment, the administration is via the intravenous or intramuscular route. The vector of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical composition of the invention locally to the area in need of treatment, e.g. the liver or the muscle. This may be achieved, for example, by means of an implant, said implant being of a porous, nonporous, or gelatinous material, including membranes, such as sialastic membranes, or fibers.

The amount of the vector of the invention which will be effective in the treatment of disorder to be treated can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the vector of the invention administered to the subject in need thereof will vary based on several factors including, without limitation, the route of administration, the specific disease treated, the subject's age or the level of expression necessary to obtain the therapeutic effect. One skilled in the art can readily determine, based on its knowledge in this field, the dosage range required based on these factors and others. In case of a treatment comprising administering an AAV vector to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg or at least 1×10¹⁵ vg/kg.

In a particular embodiment, the vector of the invention may be administered at a dose lower than typical doses used in gene therapy. In particular, in a treatment comprising administering an AAV vector to the subject in need thereof, the vector may be administered at a dose at least 2-times lower than the above typical doses, in particular at a dose at least 3-times, 4-times, 5-times, 6-times, 7-times, 8-times, 9-times, 10-times, 11-times, 12-times, 13-times, 14-times, 15-times, 16-times, 17-times, 18-times, 19-times, 20-times, 21-times, 22-times, 23-times, 24-times, 25-times, 26-times, 27-times, 28-times, 29-times, 30-times, 31-times, 32-times, 33-times, 34-times, 35-times, 36-times, 37-times, 38-times, 39-times, 40-times, 41-times, 42-times, 43-times, 44-times, 45-times, 46-times, 47-times, 48-times, 49-times, or even at least 50-times lower than the typical AAV doses typically used in gene therapy.

### EXAMPLES

### Materials and Methods

### AAV Vector Production

The AAV vectors used in this study were produced using an adenovirus-free transient transfection method of HEK293 cells and purified by Akta. Titers of AAV vector stocks were determined using qPCR. All vector preparations used in the study were titered side by side before use. The primers used for qPCR on the AAV genome annealed ITR SEQ or to codon-optimized hGAA transgene sequence: forward: 5′-agatacgccggacattggactg-3′; reverse, 5′-agatacgccggacattggactg-3′

### In Vivo Studies

Mouse studies were performed according to the French and European legislation regarding animal care and experimentation (2010/63/EU) and approved by the local institutional ethical committee. Wild-type male C57BL/6 mice were purchased from Charles River Laboratories. Gaa knockout mice (Gad) were purchased from The Jackson Laboratory (B6; 129-Gaatm1Rabn/J, stock number 004154, 6neo) and were originally generated by Raben et al. 95 Littermate male mice were used, either affected (Gaa^{-/-}) or healthy (Gaa^{+/+}). AAV vectors were delivered to adult mice via the tail vein in a volume of 0.2 mL. One month after injection, mice were sacrifice to harvest blood and tissues. Mouse experimental groups were sized at n=4 based on data generated in a previous study; all samples and animals analyzed were included in the data, and none of the outliers were excluded.

### GAA Activity Assay

Snap-frozen tissues were homogenized in UltraPure DNase- and RNase-free distilled water (Thermo Fisher Scientific). Tissues were weighed, homogenized, and centrifuged for 10 min at 10,000 g to collect the supernatant. The enzymatic reaction was set up using 10 μL of sample (plasma or tissue homogenate) diluted appropriately and 20 μL of substrate, 4-methylumbelliferone (4MU) a-D-glucoside, in black 96-well plates (PerkinElmer). The reaction mixture was incubated at 37 C for 1 hour and then stopped by adding 150 μL of sodiumcarbonate buffer (pH 10.5). A standard curve (0-2,500 pmol/mL of 4MU) was used to measure released fluorescent 4MU from the individual reaction mixture using the EnSpire Alpha plate reader (PerkinElmer) at 449 nm (emission) and 360 nm (excitation). The protein concentration of the clarified supernatant was quantified by BCA (Thermo Fisher Scientific). To calculate the GAA activity in tissues, the released 4MU concentration was divided by the sample protein concentration, and activity was reported as nanomoles per hour per milligram protein or millilitre of sera.

### Western Blot Analysis

Western blot on mouse plasma was performed on samples diluted 1:4 in distilled water. Homogenates of mouse tissues were prepared as indicated for GAA activity. Protein concentration was determined using the BCA protein assay (Thermo Fisher Scientific). SDS-PAGE electrophoresis was performed in a 4%-12% polyacrylamide gel. After transfer, the membrane was blocked with Odyssey buffer (LI-COR Biosciences) and incubated with an anti-GAA antibody (rabbit monoclonal, clone EPR4716 (2), Abcam), and anti-Gapdh (rabbit polyclonal, PA1-988, Thermo Fisher Scientific). The membrane was washed and incubated with the appropriate secondary antibody (LI-COR Biosciences) and visualized with the Odyssey imaging system (LI-COR Biosciences). For western blot quantification, we used either ImageJ or Image Studio Lite 4.0. The quantification of the hGAA protein bands in mouse tissues was normalized using either Gapdh bands. The quantification of the hGAA protein band in plasma was normalized using a nonspecific band detected by the anti-hGAA antibody in mouse plasma (used as a loading control).

### Anti-GAA and Anti-Capsid Antibody Detection

Anti-hGAA IgG capture assays were performed in a Maxisorp 96-well plates (Thermo Fisher Scientific) were coated with 2 mg/mL of rhGAA. IgG standard curves were made by serial 1 to 2 dilution of commercial mouse recombinant IgGs (Sigma-Aldrich) that were coated directly onto the wells in duplicate (from 1 mg/mL to 0.15 mg/mL). Plasma samples appropriately diluted in 10 mM PBS (pH 7.4) containing 2% BSA were analyzed in duplicate. An HRP-conjugated anti-mouse IgG antibody (human ads-HRP, Southern Biotech) was used as a secondary antibody. Plates were revealed with OPD substrate (o-phenylenediaminedihydrochloride, Sigma). The reaction was stopped with H2SO4 3 M solution, and optical density (OD) measurements were done at 492 nm using a microplate reader (ENSPIRE, PerkinElmer, Waltham, USA). Anti-AAV IgG concentration was determined against the standard curve.

mSeAP Quantification

Snap-frozen tissues were homogenized in UltraPure DNase- and RNase-free distilled water (Thermo Fisher Scientific). Tissues were weighed, homogenized, and centrifuged for 10 min at 10,000 g to collect the supernatant. The enzymatic reaction was set up using LifeTech T1015 mseap kit. Briefly, 10 μL of heated sample (plasma or tissue homogenate) were incubated 5 minutes with 10 μL of assay buffer then 20 minutes with 10 μL of reaction buffer. A standard curve (0-6 ng/μL of mSEAP) was used to measure released luminescence from the individual reaction mixture using the EnSpire Alpha plate reader (PerkinElmer). The protein concentration of the clarified supernatant was quantified by BCA (Thermo Fisher Scientific). To calculate the mSeAP expression in tissues.

### Vector Genome Copy Number Analysis

DNA was extracted from tissues homogenates using NucleoMag Pathogen (Macherey-Nagel, France) and quantified. Vector genome copy number was determined by qPCR using 500 ng of DNA, primers, and a probe annealed on ITR or on the codon-optimized hGAA (forward, 5′-agatacgccggacattggactg-3′; reverse, 5′-agatacgccggacattggactg-3′; probe, 5′-gtgtggtcctcttgggagc-3′). and mouse Titin as a reference gene (forward: 5′-aaaacgagcagtgacgtgagc-3′; reverse: 5′-ttcagtcatgctgctagcgc-3′; probe, 5′-tgcacggaagcgtctcgtctcagt-3′). The qPCR was performed using the TaqMan method.

### Statistical Analysis

All data shown in the present manuscript are reported as mean±SD. The number of sampled units, n, upon which we reported statistics, is the single mouse for the in vivo experiments (one mouse is n=1). GraphPad Prism 7.0 software was used for statistical analyses. We assessed the normal distribution of the data obtained from the different measurements (anti-hGAA IgG amounts in plasma, hGAA protein expression in plasma and tissues, GAA enzyme activity in tissues) using the Shapiro-Wilk test. The statistical tests used were unpaired Student's t test for two-group comparisons, one-way ANOVA with Tukey post hoc for comparisons of more than two groups. For all datasets analyzed by parametric tests, alpha=0.05. All statistical tests were performed two-sided. p<0.05 was considered significant. The statistical analysis performed for each dataset is indicated in the figure legends. For all figures, *p<0.05, ** p<0.01, *** p<0.001, **** p<0.0001, #p<0.05, ##p<0.01, ## #p<0.001, ## ##p<0.0001.

### Results

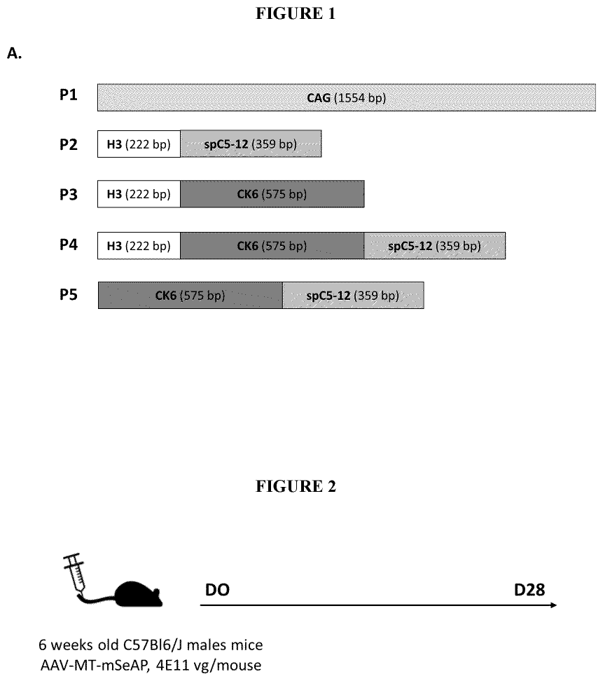

In FIG. 1A are represented the ubiquitous CAG promoter (called “P1”) and 4 different combinations of (i) H3 enhancer and spC5-12 promoter (P2), (ii) H3 enhancer and CK6 promoter (P3), (iii) H3 enhancer, CK6 promoter and spC5-12 promoter (P4) and (iv) CK6 promoter and spC5-12 promoter (P5). H3 enhancer corresponds to three repetitions of the HS-CRM8 enhancer. The sequence of P4 is as shown in SEQ ID NO:30.

In FIG. 2 is described the in vivo protocol. Six week-old C57BL/6 wild type (WT) mice were intravenously injected with 4E11 vg/mouse of an AAV-MT vector (AAV9-rh74-P1 vector) encoding murine secreted alkaline phosphatase (mSeAP) under the transcriptional control of P1, P3 or P4. One month after injection, mice were sacrificed and tissues were analyzed.

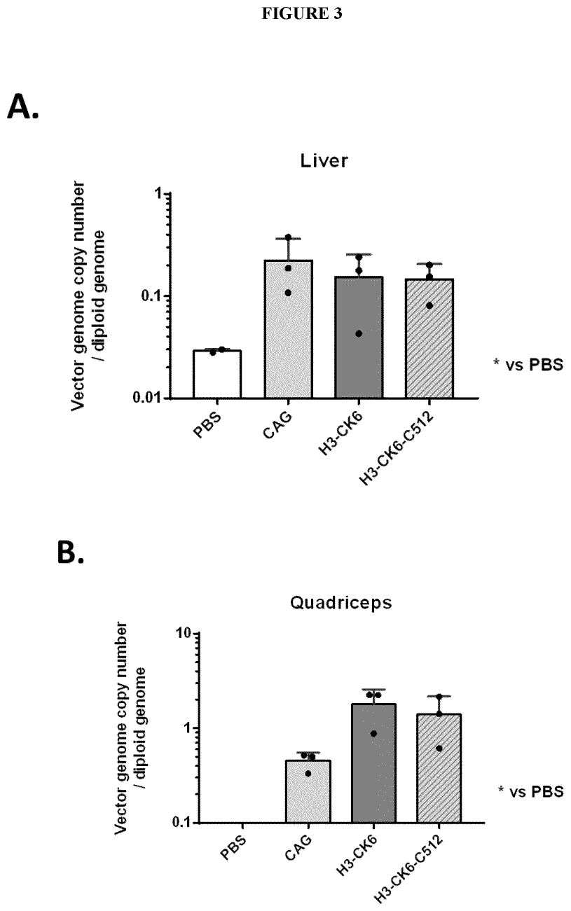

FIG. 3 represents the number of copy of transgene per cell in liver and quadriceps. Data show that both tissues were transduced with a similar efficacy by the three AAV vectors.

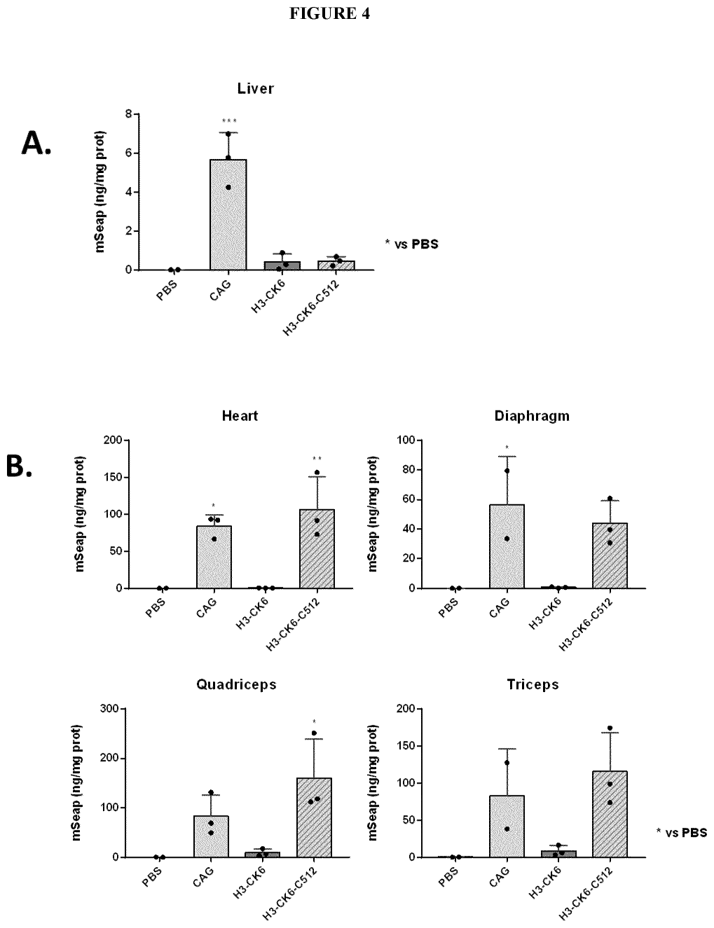

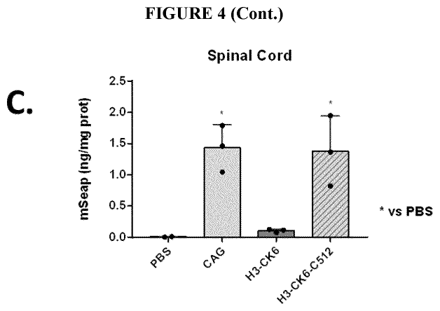

In FIG. 4 is shown the mSEAP activity measured in liver, in four different muscles (heart, diaphragm, quadriceps and triceps) and in spinal cord of mice injected with the vectors. Improved mSEAP activity was observed after transduction with the AAV-MT vector expressing mSEAP under the control of P1 and P4 in different muscles and spinal cord. Only P1 (ubiquitous promoter) expressed mSEAP in liver.

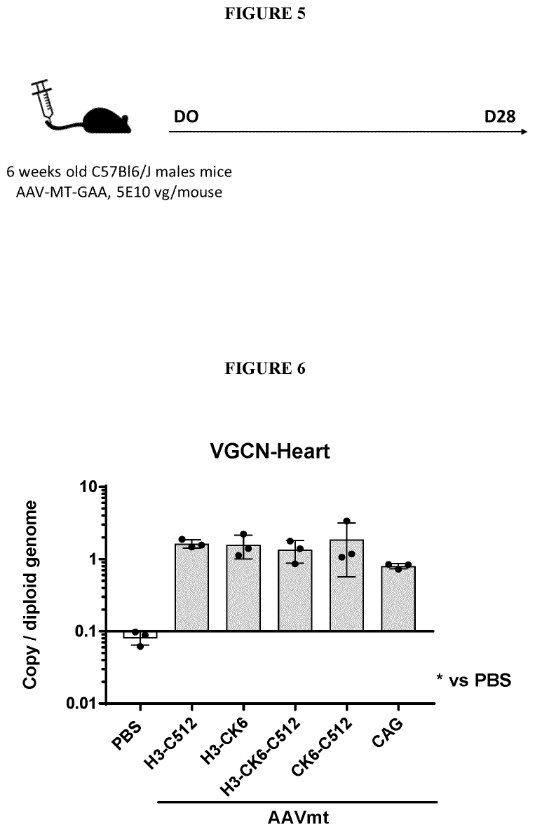

In FIG. 5 is described the in vivo protocol. Six week-old C57BL/6 wild type (WT) mice were intravenously injected with 5E10 vg/mouse of an AAV-MT (AAV9-rh74-P1) vector encoding human GAA codon optimized (hGAAco) under the transcriptional control of P1 to P5. One month after injection, mice were sacrificed and tissues were analysed.

In FIG. 6 is reported the in vector genome copy number (VGCN) normalize by titin quantification from DNA extracted from heart tissues at sacrifice. VGCN was not significantly different from each groups.

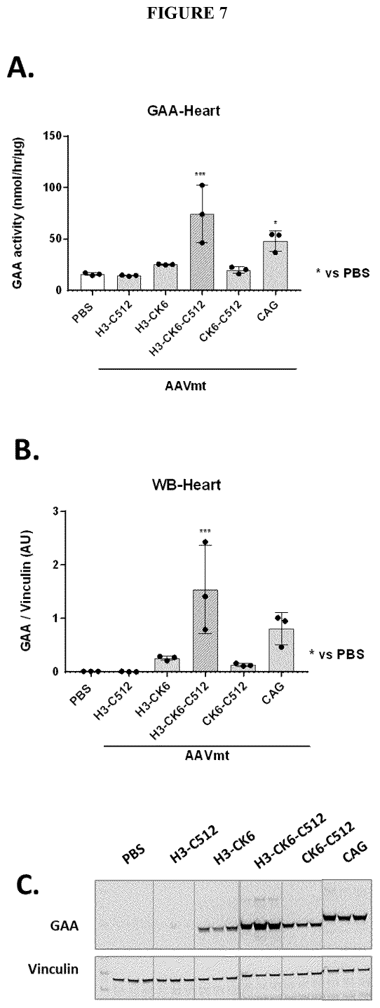

The FIG. 7 corresponds to GAA measured in heart from mice injected with the vectors encoding GAA under the transcription control of P1 to P5. FIG. 7A represents the GAA activity measured by enzymatic assay and normalized by total proteins. FIGS. 7B and 7C correspond to GAA quantification by Western Blot assay. FIG. 7B shows the quantification of GAA intensity bands normalized by Vinculin intensity bands obtained from the picture 7C. With these two methods, GAA was more expressed in mice injected with vector P4 (H3-CK6-C512) compared to the other groups.

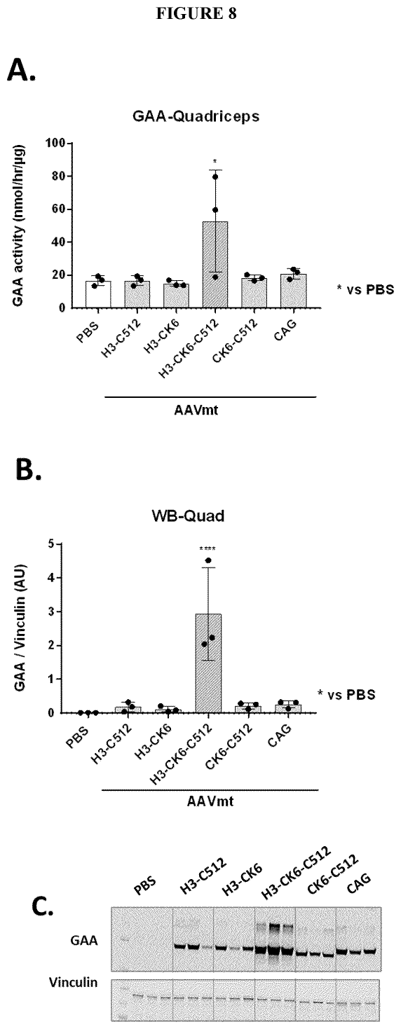

FIG. 8 corresponds to GAA measured in quadriceps from mice injected with the vectors encoding GAA under the transcription control of P1 to P5. FIG. 8A represents the GAA activity measured by enzymatic assay and normalized by total proteins. FIGS. 8B and 8C corresponds to GAA quantification by Western Blot assay. FIG. 8B shows the quantification of GAA intensity bands normalized by Vinculin intensity bands obtained from the picture 8C. With these two methods, GAA was more expressed in mice injected with vector P4 (H3-CK6-C512) compared to the other groups.

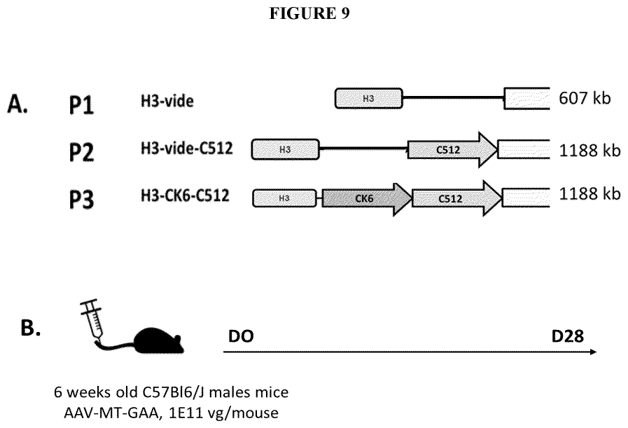

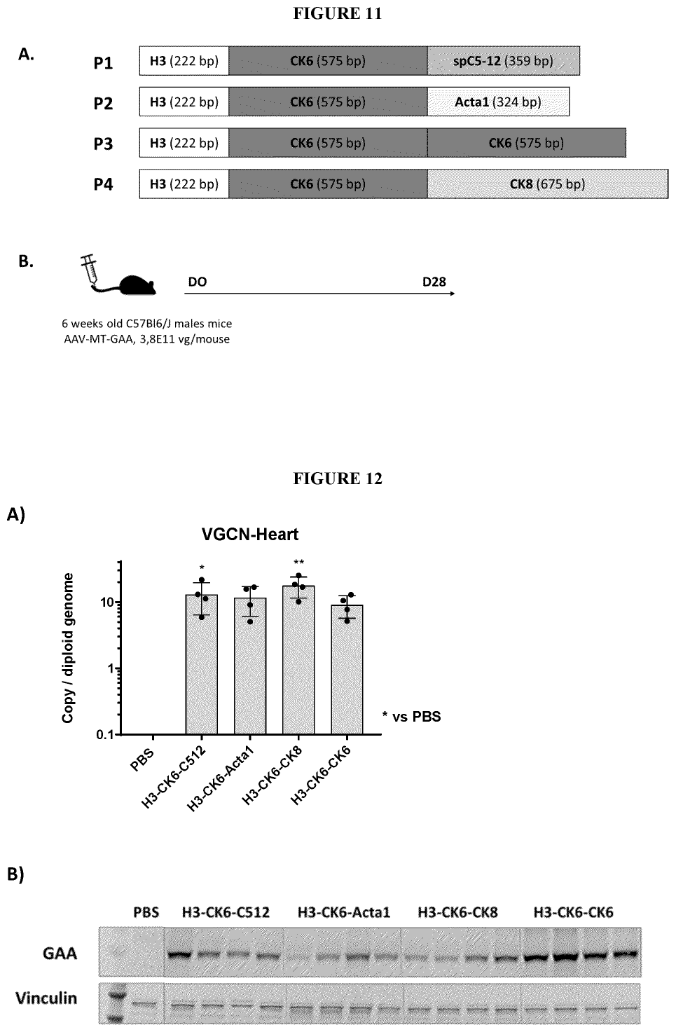

In FIG. 9A are represented 3 different combinations of H3 enhancer and a linker of 575 bp (P1); used as negative control to show the non-effect of the linker. P2 was composed of H3 enhancer; a linker used in P1. P3 correspond to H3 enhancer, CK6 and spC5-12 promoters; it was used as positive control. The sequence of P3 is as shown in SEQ ID NO:30. The FIG. 11B is described the in vivo protocol. Six week-old C57BL/6 wild type (WT) mice were intravenously injected with 1E11 vg/mouse of an AAV-MT (AAV9-rh74-P1) vector encoding human GAA codon optimized (hGAAco) under the transcriptional control of P1 to P3. One month after injection, mice were sacrificed and tissues were analysed.

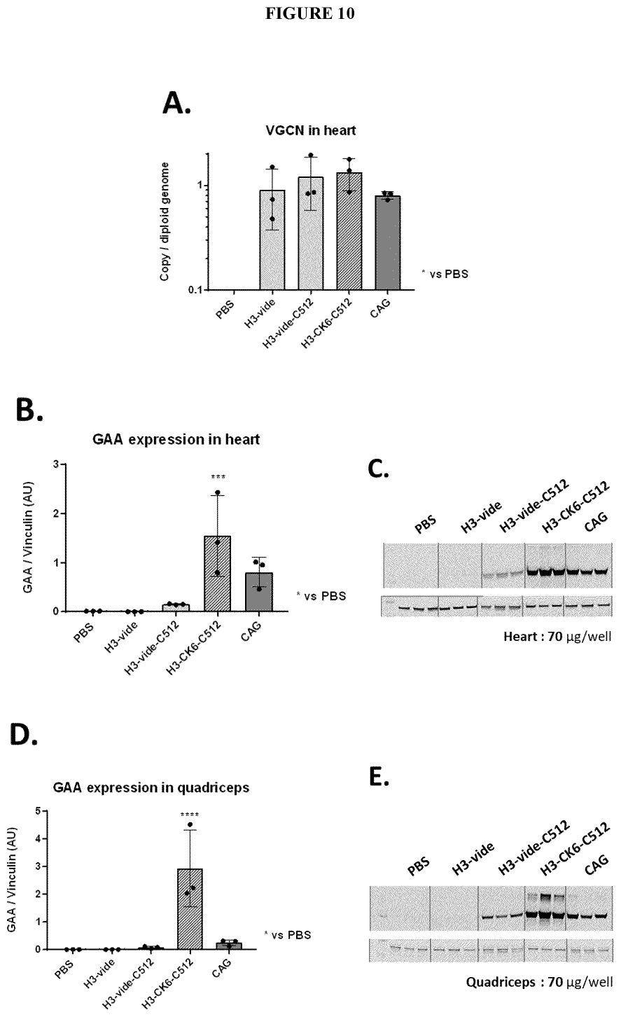

In FIG. 10A is reported the vector genome copy number (VGCN) normalized by titin quantification from DNA extracted from heart tissues at sacrifice. VGCN was not significantly different from each groups. FIG. 10B to 10E corresponds to GAA measured, by western blot, in heart (B-C) and in quadriceps (D-E) from mice injected with the vectors encoding GAA under the transcription control of P1 to P3. FIGS. 10B and 10D shows the quantification of GAA intensity bands, normalized by Vinculin intensity bands, obtained from the picture 10C and 120. GAA was more expressed in mice injected with vector P3 (H3-CK6-C512) compared to the other groups. These results demonstrate that the synergic effect of the H3-CK6-spC5-12 combination is not due to the increase in the distance between H3 and spC5-12 but to the addition of CK6 promoter between H3 and spC5-12.

In FIG. 11A are represented 4 different combinations of (i) H3 enhancer, CK6 promoter and spC5-12 promoter (P1), (ii) H3 enhancer, CK6 promoter and Acta1 promoter (P2), (iii) H3 enhancer, CK6 promoter and CK6 promoter (P3), (iv) H3 enhancer, CK6 promoter and CK8 promoter (P4). H3 enhancer corresponds to three repetitions of the HS-CRM8 enhancer. The sequence of P1 is as shown in SEQ ID NO:30. The FIG. 11B describes the in vivo protocol. Six week-old C57BL/6 wild type (WT) mice were intravenously injected with 3.8E11 vg/mouse of an AAV-MT (AAV9-rh74-P1) vector encoding human GAA codon optimized (hGAAco) under the transcriptional control of P1 to P4. One month after injection, mice were sacrificed and tissues were analysed.

In FIG. 12A is reported the vector genome copy number (VGCN) normalized by titin quantification from DNA extracted from heart tissues at sacrifice. VGCN was not significantly different from each groups. FIG. 12B corresponds to GAA measured, by western blot, in quadriceps from mice injected with the vectors encoding GAA under the transcription control of P1 to P4. The results show that similar levels of GAA expression are obtained when using P1 (H3-CK6-C5.12), P2 (H3-CK6-Acta1) or P3 (H3-CK6-CK8). P4 (H3-CK6-CK6) is also very efficient in that it seems to show an even more stronger expression, when compared P1. Said results thereby show that spC5.12 can by replaced by other muscle-specific promoters without affecting the level of transgene expression in muscles.

These data indicate that, surprisingly, the combination of liver-selective enhancers (H3) with two muscle-selective promoters (CK6+a second muscular promoter) greatly improves the expression of a protein in muscles and spinal cord (similar expression compared to a strong ubiquitous promoter) without targeting the liver.

1-15. (canceled)

16. A nucleic acid molecule comprising, operatively linked to each other:
(i) one or a plurality of liver-selective enhancer(s);
(ii) a first muscle-selective promoter, which is a CK6 promoter or a functional variant thereof; and
(iii) a second muscle-selective promoter, which is selected in the group consisting of: a spC5-12 promoter, CK6 promoter, CK8 promoter, Acta1 promoter, MCK promoter, desmin promoter, and functional variants thereof.

17. The nucleic acid molecule according to claim 16, wherein the first muscle-selective promoter is located upstream the 5′ end of the second muscle-selective promoter.

18. The nucleic acid molecule according to claim 16 comprising, in this order from 5′ to 3′:
the one or plurality of liver-selective enhancer(s);
the first muscle-selective promoter which is a CK6 promoter or a functional variant thereof; and
the second muscle-selective promoter, which is a spC5-12 promoter, CK6 promoter, CK8 promoter, Acta1 promoter or a functional variant thereof.

19. The nucleic acid molecule according to claim 16, wherein the CK6 promoter consists of the sequence shown in SEQ ID NO:7 or SEQ ID NO:35 or is a functional variant having a sequence that is at least 80% identical to SEQ ID NO:7 or SEQ ID NO:35.

20. The nucleic acid molecule according to claim 16, wherein the second muscle-selective promoter is:
the spC5-12 promoter consisting of the sequence shown in SEQ ID NO: 4, 5 or 6 or a functional variant having a sequence that is at least 80% identical to SEQ ID NO: 4, 5 or 6;
the CK8 promoter consisting of the sequence shown in SEQ ID NO:33 or SEQ ID NO:34 or a functional variant having a sequence that is at least 80% identical to SEQ ID NO:33 or SEQ ID NO: 34;
the CK6 promoter consisting of the sequence shown in SEQ ID NO:7 or SEQ ID NO:35 or a functional variant having a sequence that is at least 80% identical to SEQ ID NO:7 or SEQ ID NO: 35; or
the Acta1 promoter consisting of the sequence shown in SEQ ID NO:37, or a functional variant having a sequence that is at least 80% identical to SEQ ID NO:37.

21. The nucleic acid molecule according to claim 16, wherein the liver-selective enhancer comprises a sequence selected from the group consisting of SEQ ID NO:1, SEQ ID NO:16, SEQ ID NO:17, SEQ ID NO:18, SEQ ID NO:19, SEQ ID NO:20, SEQ ID NO: 21, SEQ ID NO:22, SEQ ID NO:23, SEQ ID NO:24, SEQ ID NO:25, SEQ ID NO:26, SEQ ID NO: 27, SEQ ID NO:28, SEQ ID NO:39, SEQ ID NO:40, SEQ ID NO:41, SEQ ID NO:42 and SEQ ID NO:43, a functional variant having 80% identity to any one of SEQ ID NO:1, SEQ ID NO: 16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO: 27 or SEQ ID NO:28; or
the plurality of liver-selective enhancers comprises at least one liver-selective enhancer comprising a sequence selected from the group consisting of SEQ ID NO:1, SEQ ID NO: 16, SEQ ID NO:17, SEQ ID NO:18, SEQ ID NO:19, SEQ ID NO:20, SEQ ID NO:21, SEQ ID NO: 22, SEQ ID NO:23, SEQ ID NO:24, SEQ ID NO:25, SEQ ID NO:26, SEQ ID NO:27, SEQ ID NO: 28, SEQ ID NO:39, SEQ ID NO:40, SEQ ID NO:41, SEQ ID NO:42 and SEQ ID NO: 43, a functional variant having 80% identity to any one of the sequences selected from SEQ ID NO: 1, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO: 26, SEQ ID NO:27, SEQ ID NO:28, SEQ ID NO:39, SEQ ID NO:40, SEQ ID NO:41, SEQ ID NO: 42 or SEQ ID NO:43.

22. The nucleic acid molecule according to claim 16, wherein all the liver-selective enhancers of the plurality of liver-selective enhancers have the same sequence.

23. The nucleic acid molecule according to claim 16, wherein the plurality of liver-selective enhancers comprises at least two liver-selective enhancers or three liver-selective enhancers.

24. The nucleic acid molecule according to claim 16, wherein the sequence of the liver-selective enhancer consists of SEQ ID NO:1, or is a functional variant having a sequence at least 80% identical to SEQ ID NO:1.

25. The nucleic acid molecule according to claim 16, wherein the nucleic acid molecule consists of SEQ ID NO:29, SEQ ID NO:30, SEQ ID NO:44, SEQ ID NO:45 or SEQ ID NO: 46, or is a functional variant having a sequence at least 80% identical to SEQ ID NO:30, SEQ ID NO:44, SEQ ID NO:45 or SEQ ID NO:46.

26. An expression cassette comprising the nucleic acid molecule according to claim 16, operably linked to a transgene of interest.

27. A vector comprising the expression cassette according to claim 26.

28. The vector according to claim 27, wherein said vector is a plasmid or viral vector.

29. The vector according to claim 27, wherein said vector is an adeno-associated virus (AAV) vector.

30. An isolated recombinant cell comprising the expression cassette according to claim 26 or a vector comprising said expression cassette.

31. A method of treating a neuromuscular disorder comprising the administration of an expression cassette according to claim 26, a vector comprising said expression cassette, or a recombinant cell comprising said expression cassette or vector to a subject having a neutromuscular disorder selected from the group consisting of: muscular dystrophies, motor neuron diseases inflammatory myopathies, diseases of neuromuscular junctions, diseases of peripheral nerve system metabolic diseases of muscle, myopathies due to endocrine abnormalities, and other myopathies.

32. The method according to claim 31, wherein said:
muscular dystrophies are selected from the group consisting of myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy;
motor neuron diseases are selected from the group consisting of amyotrophic lateral sclerosis (ALS), spinal muscular atrophy (Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4), and spinal-bulbar muscular atrophy (Kennedy disease);
inflammatory myopathies are selected from the group consisting of polymyositis dermatomyositis, and inclusion-body myositis;
diseases of neuromuscular junction are selected from the group consisting of myasthenia gravis, Lambert-Eaton (myasthenic) syndrome, and congenital myasthenic syndromes;
diseases of peripheral nerve are selected from the group consisting of Charcot-Marie-Tooth disease, Friedreich's ataxia, and Dejerine-Sottas disease;
metabolic diseases of muscle are selected from the group consisting of phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphoglycerate kinase deficiency, phosphoglycerate mutase deficiency, lactate dehydrogenase deficiency, and myoadenylate deaminase deficiency;
myopathies due to endocrine abnormalities are selected from the group consisting of hyperthyroid myopathy and hypothyroid myopathy; and
other myopathies are selected from the group consisting of myotonia congenita, paramyotonia congenita, central core disease, nemaline myopathy, myotubular myopathy and periodic paralysis.


SEQUENCE LISTING (51 sequences)

SEQ ID NO: 1 - DNA, 72 bp, synthetic construct
  misc_feature 1..72: note = HS-CRM8 (x1)
  source 1..72: mol_type = other DNA
gggggaggctgctggtgaatattaaccaaggtcaccccagttatcggaggagcaaacaggggctaagtccac

SEQ ID NO: 2 - DNA, 222 bp, synthetic construct
  misc_feature 1..222: note = HS-CRM8 (x3)with linker
  source 1..222: mol_type = other DNA
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccac

SEQ ID NO: 3 - DNA, 216 bp, synthetic construct
  misc_feature 1..216: note = HS-CRM8 (x3) without linker
  source 1..216: mol_type = other DNA
gggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccac

SEQ ID NO: 4 - DNA, 315 bp, synthetic construct
  misc_feature 1..315: note = spC5.12 (1)
  source 1..315: mol_type = other DNA
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 5 - DNA, 400 bp, synthetic construct
  misc_feature 1..400: note = spC5.12 (2)
  source 1..400: mol_type = other DNA
ccgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatat

SEQ ID NO: 6 - DNA, 359 bp, synthetic construct
  misc_feature 1..359: note = spC5.12 (3)
  source 1..359: mol_type = other DNA
ccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 7 - DNA, 575 bp, synthetic construct
  misc_feature 1..575: note = CK6 promoter
  source 1..575: mol_type = other DNA
ctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 8 - DNA, 10 bp, synthetic construct
  misc_feature 1..10: note = linker
  source 1..10: mol_type = other DNA
ttaatgaccc

SEQ ID NO: 9 - DNA, 91 bp, synthetic construct
  misc_feature 1..91: note = SV40 intron
  source 1..91: mol_type = other DNA
ctctaaggtaaatataaaatttttaagtgtataatgtgttaaactactgattctaattgtttgtgtattttagattccaacctatggaact

SEQ ID NO: 10 - DNA, 441 bp, synthetic construct
  misc_feature 1..441: note = HBB2 intron
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 11 - DNA, 441 bp, synthetic construct
  misc_feature 1..441: note = modified HBB2 intron
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 12 - DNA, 1438 bp, synthetic construct
  misc_feature 1..1438: note = FIX intron
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 13 - DNA, 1438 bp, synthetic construct
  misc_feature 1..1438: note = modified FIX intron
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 14 - DNA, 881 bp, synthetic construct
  misc_feature 1..881: note = Chicken beta globin intron
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 15 - DNA, 881 bp, synthetic construct
  misc_feature 1..881: note = modified Chicken beta globin intron
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 16 - DNA, 101 bp, synthetic construct
  misc_feature 1..101: note = HS-CRM1
  source 1..101: mol_type = other DNA
cagccaatgaaatacaaagatgagtctagttaataatctacaattattggttaaagaagtatattagtgctaatttccctccgtttgtcctagcttttctc

SEQ ID NO: 17 - DNA, 71 bp, synthetic construct
  misc_feature 1..71: note = HS-CRM2
  source 1..71: mol_type = other DNA
tgaatgaccttcagcctgttcccgtccctgatatgggcaaacattgcaagcagcaaacagcaaacacatag

SEQ ID NO: 18 - DNA, 173 bp, synthetic construct
  misc_feature 1..173: note = HS-CRM3
  source 1..173: mol_type = other DNA
ggcgtattcttaagaatagattaaataatcataaaaagatctatacttaaaaattgaaaaatgcttaaatattaaaattcttctcataaaaaaatactaatttaaaaatgagcctgaaatgtttatctatttattgcacagggttgcatacataaaacgacacaccctcttgt

SEQ ID NO: 19 - DNA, 551 bp, synthetic construct
  misc_feature 1..551: note = HS-CRM4
  source 1..551: mol_type = other DNA
agtttggaacaagactatataccatatcctacaggaagaataaaagtaaaggaaaggtgccatctctactgaatagagagtcctaacaaaaaggcttcaaaaggactctgcatctttaataatataaaaaggctaggacacaaacagcatcatctaaaatgccattagaaatacttcacatacaaaaaggtctaagtaaagcaggattttataaagtgatcaaaaaagaaacactaagggggaaaaatcttttaagattaaagaggtttttcaaaggacaagttgaagtggctgtaaaatttatgaggcagcattaaacttcagttctaagtaacaataaattattcaccataaaaacatacatgtgtcaaatattataagcctcttaaactttttaaaacaatttcttgcagaactgattagatatattaagtcaagattagcagatactaactttttcattagcatactatgatcactcagagtaaaggaggaaatttagaaaagaaataagacagaaccatcaatagtcgattcaccaccaaatgtga

SEQ ID NO: 20 - DNA, 141 bp, synthetic construct
  misc_feature 1..141: note = HS-CRM5
  source 1..141: mol_type = other DNA
tgcgggaatcagcctttgaaacgatggccaacagcagctaataataaaccagtaatttgggatagacgagtagcaagagggcattggttggtgggtcaccctccttctcagaacacattataaaaaccttccgtttccaca

SEQ ID NO: 21 - DNA, 135 bp, synthetic construct
  misc_feature 1..135: note = HS-CRM6
  source 1..135: mol_type = other DNA
gcatgattttaaggactggttgtttatgagccaatcagaggtgttgaataaacacctccctactaggtcaaggtagaaaggggagggcaaatattggaaaaaaaaaacatgatgagaagtctataaaaattgtgt

SEQ ID NO: 22 - DNA, 94 bp, synthetic construct
  misc_feature 1..94: note = HS-CRM7
  source 1..94: mol_type = other DNA
ctaaaatgggcaaacattgcaagcagcaaacagcaaacacacagccctccctgcctgctgaccttggagctggggcagaggtcagagacctctc

SEQ ID NO: 23 - DNA, 171 bp, synthetic construct
  misc_feature 1..171: note = HS-CRM9
  source 1..171: mol_type = other DNA
aggaggaactgctcaaaacagacagaggctctttgtttgctttgcttctgtgtcaactgggcaacatttggaaacaacaaatattggttcagaggcccactgctttcttacccacctcctgctggtcagcttttccagctttcctgcacgtacacacaagcgcagctattt

SEQ ID NO: 24 - DNA, 170 bp, synthetic construct
  misc_feature 1..170: note = HS-CRM10
  source 1..170: mol_type = other DNA
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 25 - DNA, 74 bp, synthetic construct
  misc_feature 1..74: note = HS-CRM11
  source 1..74: mol_type = other DNA
tgccactcctagttcccatcctatttaaatctgcaagaggtttggttaatcattggctttgtcctgtgtagaca

SEQ ID NO: 26 - DNA, 441 bp, synthetic construct
  misc_feature 1..441: note = HS-CRM12
  source 1..441: mol_type = other DNA
ttccttcccccttccaagacccccctgaatcctatcaaaagcacatcttccattcattgcttcccggtgtcattatgacaagcggctacaaatcaatagcagagggaaaggcaggaccaacccgcactcaccaagtgataaagattcactctcagccccgatttgctaatagcccataatagcagccattggcgccccgcattaaataatacatttcactccgcgtttattatgggatttttaaaactcctcaccaaattggattttctcgatggtctctaatttccacatttatcatttaaaattaaactgctctgtggaaaggggggatagagaagaagaaggtagagagaggccagacagtactgtatttttccttttgactcccccctttatgaaaacccataaataatatcaggtatcacagctataagcagcagg

SEQ ID NO: 27 - DNA, 88 bp, synthetic construct
  misc_feature 1..88: note = HS-CRM13
  source 1..88: mol_type = other DNA
ggagttgctggtgcttccccaggctggagattgagttaatattaacaggcccaaggcgatgtgggcttgtgcaatcataggcccggcc

SEQ ID NO: 28 - DNA, 41 bp, synthetic construct
  misc_feature 1..41: note = HS-CRM14
  source 1..41: mol_type = other DNA
atcgccaggtcacctgaggagttaatgaatacatatctcct

SEQ ID NO: 29 - DNA, 1171 bp, synthetic construct
  misc_feature 1..1171: note = H3 - CK6 - spC5.12(1)
  source 1..1171: mol_type = other DNA
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttaatgaccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 30 - DNA, 1166 bp, synthetic construct
  misc_feature 1..1166: note = H3-CK6-C512 (2)
  source 1..1166: mol_type = other DNA
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgcgccctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 31 - DNA, 1394 bp, synthetic construct
  misc_feature 1..1394: note = H3-CK6-H3-C512
  source 1..1394: mol_type = other DNA
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgcgccctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttccgggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgcgccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 32 - DNA, 1166 bp, synthetic construct
  misc_feature 1..1166: note = CK6-H3- C512
  source 1..1166: mol_type = other DNA
ctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttccgggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgcgccccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 33 - DNA, 454 bp, synthetic construct
  misc_feature 1..454: note = CK8 promoter
  source 1..454: mol_type = other DNA
ctacaaacgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 34 - DNA, 338 bp, synthetic construct
  misc_feature 1..338: note = short CK8 promoter
  source 1..338: mol_type = other DNA
ctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 35 - DNA, 377 bp, synthetic construct
  misc_feature 1..377: note = short CK6 promoter
  source 1..377: mol_type = other DNA
cctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 36 - DNA, 366 bp, synthetic construct
  misc_feature 1..366: note = MCK promoter
  source 1..366: mol_type = other DNA
caatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 37 - DNA, 324 bp, synthetic construct
  misc_feature 1..324: note = Acta1 promoter
  source 1..324: mol_type = other DNA
aaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 38 - DNA, 1060 bp, synthetic construct
  misc_feature 1..1060: note = Desmin promoter
  source 1..1060: mol_type = other DNA
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 39 - DNA, 321 bp, synthetic construct
  source 1..321: mol_type = other DNA
  misc_feature 1..321: note = Apo-E enhancer
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 40 - DNA, 550 bp, synthetic construct
  source 1..550: mol_type = other DNA
  misc_feature 1..550: note = enhancer A3
gtttggaacaagactatataccatatcctacaggaagaataaaagtaaaggaaaggtgccatctctactgaatagagagtcctaacaaaaaggcttcaaaaggactctgcatctttaataatataaaaaggctaggacacaaacagcatcatctaaaatgccattagaaatacttcacatacaaaaaggtctaagtaaagcaggattttataaagtgatcaaaaaagaaacactaagggggaaaaatcttttaagattaaagaggtttttcaaaggacaagttgaagtggctgtaaaatttatgaggcagcattaaacttcagttctaagtaacaataaattattcaccataaaaacatacatgtgtcaaatattataagcctcttaaactttttaaaacaatttcttgcagaactgattagatatattaagtcaagattagcagatactaactttttcattagcatactatgatcactcagagtaaaggaggaaatttagaaaagaaataagacagaaccatcaatagtcgattcaccaccaaatgtga

SEQ ID NO: 41 - DNA, 74 bp, synthetic construct; identical to SEQ ID NO: 25
  source 1..74: mol_type = other DNA
  misc_feature 1..74: note = enhancer F

SEQ ID NO: 42 - DNA, 88 bp, synthetic construct; identical to SEQ ID NO: 27
  source 1..88: mol_type = other DNA
  misc_feature 1..88: note = enhancer S1

SEQ ID NO: 43 - DNA, 41 bp, synthetic construct; identical to SEQ ID NO: 28
  source 1..41: mol_type = other DNA
  misc_feature 1..41: note = enhancer S2

SEQ ID NO: 44 - DNA, 1269 bp, synthetic construct
  source 1..1269: mol_type = other DNA
  misc_feature 1..1269: note = H3-CK6-CK8
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtcctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttaatgggactctacaaacgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 45 - DNA, 1390 bp, synthetic construct
  source 1..1390: mol_type = other DNA
  misc_feature 1..1390: note = H3-CK6-CK6
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtcctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttaatgggactctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 46 - DNA, 1138 bp, synthetic construct
  source 1..1138: mol_type = other DNA
  misc_feature 1..1138: note = H3-CK6-Acta1
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtcctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacttaatgggacaaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 47 - DNA, 22 bp, synthetic construct
  source 1..22: mol_type = other DNA
agatacgccggacattggactg

SEQ ID NO: 48 - DNA, 19 bp, synthetic construct
  source 1..19: mol_type = other DNA
gtgtggtcctcttgggagc

SEQ ID NO: 49 - DNA, 21 bp, synthetic construct
  source 1..21: mol_type = other DNA
aaaacgagcagtgacgtgagc

SEQ ID NO: 50 - DNA, 20 bp, synthetic construct
  source 1..20: mol_type = other DNA
ttcagtcatgctgctagcgc

SEQ ID NO: 51 - DNA, 24 bp, synthetic construct
  source 1..24: mol_type = other DNA
tgcacggaagcgtctcgtctcagt